SEQUENCE LISTING (9 sequences)

SEQ ID NO: 1 - RNA, 454 nt, Artificial Sequence
  Synthetic nucleotide sequence derived from HCV IRES
gccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaacguaacaccaaccgccgcccacaggacgucugaggcggccgccgccucagacguccuguggguuuauugcaucccgc

SEQ ID NO: 2 - DNA, 489 bp, Artificial Sequence
  Synthetic nucleotide sequence derived from HCV IRES, with T7promoter and primer annealing site
ggatcctaatacgactcactatagccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtctgaggcggccgccgcctcagacgtcctgtgggtttattgcatcccgcctcgagtctaga

SEQ ID NO: 3 - RNA, 574 nt, Artificial Sequence
  Synthetic nucleotide sequence derived from Trichoderma atroviride FgenI
ucucucgcuaucucggaaucgaggggucuggccuacgcugcgauugcuucuucagagcauccuucuucagccuugugcccucuacaguggcagccucacacaaacuaucaacauggcuuccgaauccaggcucuaccagaucuccggcgagaccaagucucaucugcucaaguuccgcuugacgacaucucgggccagcaaaccucaggcagugauuuauuugauugauaaaaacacucacgaaauccgccaagacgaugacaagacuguauacaccucccucgacgagauagccgacgaccuccccgacagcaccccucgauucaucuuccucagcuaucccuugacgaugcccgauggacggcuauccgugccuuacaccaugaucuacuaccuccccaucaacugcaaugccgcaacaaggaugcucuacgccggcgcaaaagagcugauacgcaacacggccgaggugaacaagguuaucgauauagagucugcagaggaucuggaagauauuccaaagcaguugagcggauagaaauaauaauaaaagaaauaacauguucgcuaugcc

SEQ ID NO: 4 - RNA, 612 nt, Artificial Sequence
  Synthetic nucleotide sequence derived from Trichoderma atroviride FgenI, with primer annealing sites
uccaccaugaaucacuccucucucgcuaucucggaaucgaggggucuggccuacgcugcgauugcuucuucagagcauccuucuucagccuugugcccucuacaguggcagccucacacaaacuaucaacauggcuuccgaauccaggcucuaccagaucuccggcgagaccaagucucaucugcucaaguuccgcuugacgacaucucgggccagcaaaccucaggcagugauuuauuugauugauaaaaacacucacgaaauccgccaagacgaugacaagacuguauacaccucccucgacgagauagccgacgaccuccccgacagcaccccucgauucaucuuccucagcuaucccuugacgaugcccgauggacggcuauccgugccuuacaccaugaucuacuaccuccccaucaacugcaaugccgcaacaaggaugcucuacgccggcgcaaaagagcugauacgcaacacggccgaggugaacaagguuaucgauauagagucugcagaggaucuggaagauauuccaaagcaguugagcggauagaaauaauaauaaaagaaauaacauguucgcuaugccgaaaaaccaaacguaacacc

SEQ ID NO: 5 - DNA, 612 bp, Artificial Sequence
  Synthetic nucleotide sequence derived from Trichoderma atroviride FgenI, with primer annealing sites
tccaccatgaatcactcctctctcgctatctcggaatcgaggggtctggcctacgctgcgattgcttcttcagagcatccttcttcagccttgtgccctctacagtggcagcctcacacaaactatcaacatggcttccgaatccaggctctaccagatctccggcgagaccaagtctcatctgctcaagttccgcttgacgacatctcgggccagcaaacctcaggcagtgatttatttgattgataaaaacactcacgaaatccgccaagacgatgacaagactgtatacacctccctcgacgagatagccgacgacctccccgacagcacccctcgattcatcttcctcagctatcccttgacgatgcccgatggacggctatccgtgccttacaccatgatctactacctccccatcaactgcaatgccgcaacaaggatgctctacgccggcgcaaaagagctgatacgcaacacggccgaggtgaacaaggttatcgatatagagtctgcagaggatctggaagatattccaaagcagttgagcggatagaaataataataaaagaaataacatgttcgctatgccgaaaaaccaaacgtaacacc

SEQ ID NO: 6 - RNA, 1667 nt, Artificial Sequence
  Synthetic nucleotide sequence derived from Trichoderma atroviride Chi18-4
uacgccagacaauagauucagcucgaauaaugaagccgauuggucacugauuaacuugguguaucuacuggguagcaaaggaccgaucuucaccucgcaugaccguugaauguggcgcaauucuaguacuugaauggccuggccaagcuuauagaacaaucgagacgaauugaacaccacuaucaaaauccaaauuaugcgguuuucaaucauacauguggcuuuauggcuggccauggcggccgguucauuuacagcuaacgcuuacggagcuguucgaugcgucauguaucucacagggcaacacgugguagucccuucagaaccucaucucguggauuccauaacccauuugauacuggcuuuuaugcgcucugaugucuucaauguggacaaaacgccugcugaauucccgcucuucgcaucaguugcugagacgcgcgagaaguucaacgcaaauaccaagaucauggucgcaaucggcgguuggggagacgcaggauuugaagaagcugcgcgugacgauucaucgagaaagcggugggcuggccacgucaaggccaugguugaccagacaggagccgauggcauugacauugacugggaauauccgggagggaaccgugacgauuauaaacucauuccgaacucucagcgggaaugggagauucaggcauucgugcuccuucuucaagaauugcguuccgccuugggacaaacgaaacugcucucaauugcggugccagcgcuggaacgagauuugauggccuucacaaacucgacuauuccguccauuguggaucaaguugacuuuaucaguguaaugacuuacgacaugaugaaccgacgcgacaacguugucaagcaccacagcggcguggcugacuccugggaagcaaugaagcgauauauagaucgcggcgucccuccgcacaagcugaacuuuggacuugguuacuaugccaaaugguucaugacugaacaaugcgauguacagcacccauuaggaugccgcacucaacugcuagaagaccccgccaauggagccgaucuuggcaagacugcagcuuuuagcuggcacgacgagguucccgcagaaauggccaauuccuuuggcaaagcccaugcucauggccgcuacuaugaagauggaagcuacggcuauugggacgaugaagagaagagauggugguccuacgacacgccucucaccaucaaagccaaagugccucggcuucucggcgagcugcagcugggcgggguguuugccugggggcugggcgaggacgcuccgcaguuucugcaccugaaggcgacuguugacggcauucggguucugcgcggagaugacgaugcugugaaggaugagcuguaacaagcagcggcguguguacgucuggaguuuggcgacaucauugaaggcgugggaugcaauucaagccauugauacugcagauaaguugaagcagcaacagccaaaacucugcacguggcccgagacuuccgucuuauagcagggaacagacaggauggugcugacggaauaugcagagcucgagcucaaccgguaggcuagcuugaaaaccccaugagcuagcaguuaaccuugcaguuaaggguaaccagagcuuaaacacgucugauuuggugcaaugaauacaggcugc

SEQ ID NO: 7 - RNA, 1705 nt, Artificial Sequence
  Synthetic nucleotide sequence derived from Trichoderma atroviride Chi18-4, with primer annealing sites
uccaccaugaaucacuccuacgccagacaauagauucagcucgaauaaugaagccgauuggucacugauuaacuugguguaucuacuggguagcaaaggaccgaucuucaccucgcaugaccguugaauguggcgcaauucuaguacuugaauggccuggccaagcuuauagaacaaucgagacgaauugaacaccacuaucaaaauccaaauuaugcgguuuucaaucauacauguggcuuuauggcuggccauggcggccgguucauuuacagcuaacgcuuacggagcuguucgaugcgucauguaucucacagggcaacacgugguagucccuucagaaccucaucucguggauuccauaacccauuugauacuggcuuuuaugcgcucugaugucuucaauguggacaaaacgccugcugaauucccgcucuucgcaucaguugcugagacgcgcgagaaguucaacgcaaauaccaagaucauggucgcaaucggcgguuggggagacgcaggauuugaagaagcugcgcgugacgauucaucgagaaagcggugggcuggccacgucaaggccaugguugaccagacaggagccgauggcauugacauugacugggaauauccgggagggaaccgugacgauuauaaacucauuccgaacucucagcgggaaugggagauucaggcauucgugcuccuucuucaagaauugcguuccgccuugggacaaacgaaacugcucucaauugcggugccagcgcuggaacgagauuugauggccuucacaaacucgacuauuccguccauuguggaucaaguugacuuuaucaguguaaugacuuacgacaugaugaaccgacgcgacaacguugucaagcaccacagcggcguggcugacuccugggaagcaaugaagcgauauauagaucgcggcgucccuccgcacaagcugaacuuuggacuugguuacuaugccaaaugguucaugacugaacaaugcgauguacagcacccauuaggaugccgcacucaacugcuagaagaccccgccaauggagccgaucuuggcaagacugcagcuuuuagcuggcacgacgagguucccgcagaaauggccaauuccuuuggcaaagcccaugcucauggccgcuacuaugaagauggaagcuacggcuauugggacgaugaagagaagagauggugguccuacgacacgccucucaccaucaaagccaaagugccucggcuucucggcgagcugcagcugggcgggguguuugccugggggcugggcgaggacgcuccgcaguuucugcaccugaaggcgacuguugacggcauucggguucugcgcggagaugacgaugcugugaaggaugagcuguaacaagcagcggcguguguacgucuggaguuuggcgacaucauugaaggcgugggaugcaauucaagccauugauacugcagauaaguugaagcagcaacagccaaaacucugcacguggcccgagacuuccgucuuauagcagggaacagacaggauggugcugacggaauaugcagagcucgagcucaaccgguaggcuagcuugaaaaccccaugagcuagcaguuaaccuugcaguuaaggguaaccagagcuuaaacacgucugauuuggugcaaugaauacaggcugcgaaaaaccaaacguaacacc

SEQ ID NO: 8 - DNA, 1705 bp, Artificial Sequence
  Synthetic nucleotide sequence derived from Trichoderma atroviride Chi18-4, with primer annealing sites
tccaccatgaatcactcctacgccagacaatagattcagctcgaataatgaagccgattggtcactgattaacttggtgtatctactgggtagcaaaggaccgatcttcacctcgcatgaccgttgaatgtggcgcaattctagtacttgaatggcctggccaagcttatagaacaatcgagacgaattgaacaccactatcaaaatccaaattatgcggttttcaatcatacatgtggctttatggctggccatggcggccggttcatttacagctaacgcttacggagctgttcgatgcgtcatgtatctcacagggcaacacgtggtagtcccttcagaacctcatctcgtggattccataacccatttgatactggcttttatgcgctctgatgtcttcaatgtggacaaaacgcctgctgaattcccgctcttcgcatcagttgctgagacgcgcgagaagttcaacgcaaataccaagatcatggtcgcaatcggcggttggggagacgcaggatttgaagaagctgcgcgtgacgattcatcgagaaagcggtgggctggccacgtcaaggccatggttgaccagacaggagccgatggcattgacattgactgggaatatccgggagggaaccgtgacgattataaactcattccgaactctcagcgggaatgggagattcaggcattcgtgctccttcttcaagaattgcgttccgccttgggacaaacgaaactgctctcaattgcggtgccagcgctggaacgagatttgatggccttcacaaactcgactattccgtccattgtggatcaagttgactttatcagtgtaatgacttacgacatgatgaaccgacgcgacaacgttgtcaagcaccacagcggcgtggctgactcctgggaagcaatgaagcgatatatagatcgcggcgtccctccgcacaagctgaactttggacttggttactatgccaaatggttcatgactgaacaatgcgatgtacagcacccattaggatgccgcactcaactgctagaagaccccgccaatggagccgatcttggcaagactgcagcttttagctggcacgacgaggttcccgcagaaatggccaattcctttggcaaagcccatgctcatggccgctactatgaagatggaagctacggctattgggacgatgaagagaagagatggtggtcctacgacacgcctctcaccatcaaagccaaagtgcctcggcttctcggcgagctgcagctgggcggggtgtttgcctgggggctgggcgaggacgctccgcagtttctgcacctgaaggcgactgttgacggcattcgggttctgcgcggagatgacgatgctgtgaaggatgagctgtaacaagcagcggcgtgtgtacgtctggagtttggcgacatcattgaaggcgtgggatgcaattcaagccattgatactgcagataagttgaagcagcaacagccaaaactctgcacgtggcccgagacttccgtcttatagcagggaacagacaggatggtgctgacggaatatgcagagctcgagctcaaccggtaggctagcttgaaaaccccatgagctagcagttaaccttgcagttaagggtaaccagagcttaaacacgtctgatttggtgcaatgaatacaggctgcgaaaaaccaaacgtaacacc

SEQ ID NO: 9 - RNA, 30 nt, Artificial Sequence
  Synthetic nucleotide sequence
ggaaucucucucacgaacugacguaaucuu

(19) United States
(12) Patent Application Publication
(10) Pub. No.: US 2023/0414516 A1
(43) Pub. Date: Dec. 28, 2023

(54) ENHANCED FORMULATION STABILIZATION AND IMPROVED LYOPHILIZATION PROCESSES

(71) Applicant: BioNTech SE, Mainz (DE)

(72) Inventors: Bakul Subodh Bhatnagar, North Reading, MA (US); Ramin Darvari, Lexington, MA (US); Sumit Luthra, Westford, MA (US); Serguei A. Tchessalov, Billerica, MA (US); Steffen Panzner, Halle (DE); Christian Reinsch, Halle (DE); Kaushik Thanki, Wiesbaden (DE); Sukrut Somani, Mainz (DE)

(21) Appl. No.: 18/036,676

(22) PCT Filed: Nov. 15, 2021
(86) PCT No.: PCT/EP21/81625

§ 371 Date: May 12, 2023

Related U.S. Application Data
(60) Provisional application No. 63/149,372, filed on Feb. 15, 2021.
(60) Provisional application No. 63/135,723, filed on Jan. 10, 2021.
(60) Provisional application No. 63/115,128, filed on Nov. 18, 2020.
(60) Provisional application No. 63/115,588, filed on Nov. 18, 2020.
(60) Provisional application No. 63/114,478, filed on Nov. 16, 2020.

(51) Int. Cl. A61K 9/19; A61K 9/16; A61K 9/51; A61K 31/7105

(52) U.S. Cl. CPC A61K 9/19; A61K 9/1694; A61K 9/1623; A61K 9/5123; A61K 31/7105

(57) ABSTRACT

This invention relates to enhanced stabilization and improved lyophilization methods of pharmaceutical substances.

### REFERENCE TO SEQUENCE LISTING

This application includes a sequence listing which is part of the specification and is incorporated herein by reference in its entirety.

### FIELD OF THE INVENTION

The invention relates to improved stabilization and lyophilization methods of pharmaceutical substances.

### BACKGROUND OF THE INVENTION

Lyophilization or freeze-drying is a widely used process in the pharmaceutical industry for the preservation of biological and pharmaceutical substances. In lyophilization, water present in a pharmaceutical substance is converted to ice during a freezing step and then removed from the pharmaceutical substance by direct sublimation under low-pressure conditions during a primary drying step. During freezing, however, not all of the water is transformed to ice. Some portion of the water is trapped in a matrix of solids containing, for example, formulation components and/or the active ingredient (pharmaceutical substance). Additional drying step (secondary drying) at elevated temperature is therefore required to remove residual moisture and achieve required moisture level.

Lyophilization process is closely linked to formulation (Bhatnagar, B. et al., Freeze-drying of biologics. Encyclopedia of Pharmaceutical Science and Technology, 4th edition, 2013, Swarbrick, J., Ed.; Wiley Interscience: New York, NY, USA: 1673-1722). Formulations also define the stability of the active pharmaceutical ingredient. For example, it has been shown that for the stabilization of proteins, a specific ratio of stabilizer to protein (molar ratio of greater than 360) is required to achieve room temperature stability (Cleland, J. L. et al. (2001) J Pharm Sci 90(3):310-321). It has also been shown that this ratio is specific to protein structure. Therefore, each protein potentially requires a different ratio of stabilizer to active ingredient to achieve similar stability (Wang, B., et al., (2009) J Pharm Sci 98(9):3145-3166. As it has been discussed in the literature (Chang, L., et al. (2009) J Pharm Sci 98(9):2886-2908), besides the specific interaction between a stabilizer and an active ingredient, an increase in stabilizer to protein ratio also results in a reduction of interaction between two proteins in solid state due to dilution effect.

In earlier work, an increase in the mass ratio of Sucrose to nucleic acid (e.g. DNA) by a factor of 2 resulted in a decrease in particle size by at least a factor of 3 using dynamic light scattering (Kasper, J. C., et al. (2013) J Pharm Sci 102(3):929-946).

### SUMMARY OF THE INVENTION

Therefore, it is an objective of the present invention to provide a method for stabilization of pharmaceutical substances, which is scalable, reproducible, and applicable for the production of pharmaceuticals and which is time- and cost-efficient. One object of the invention is to provide a method for lyophilization of a pharmaceutical substance, by which the integrity and the biological activity of the pharmaceutical substance is preferably maintained. It is a further object of the invention to provide a composition comprising a pharmaceutical substance, which is suitable for storage at ambient or subambient temperatures and over extended periods as a liquid, frozen liquid, or dried solid, and which preferably has increased storage stability as compared to prior art compositions.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 depicts an Alternate Fabrication Process describing the process flow for a biphasic system (aqueous and organic phases).

FIG. 2 depicts an Alternate Fabrication Process describing the process flow for a system with both phases being aqueous.

FIG. 3A-3B depicts a relationship between fill volume and drying time for two vial sizes (FIG. 3A) and the impact of cake height on the drying time for any vial size (FIG. 3B). Drying time includes freezing (with annealing), primary and secondary drying steps.

### SEQUENCE IDENTIFIERS

SEQ ID NO: 1 sets forth RNA sequence derived from HCV IRES.

SEQ ID NO: 2 sets forth DNA transcript, derived from HCV IRES (T7 promoter in bold; site in italics).

SEQ ID NO: 3 sets forth FgenI model RNA synthesized RNA sequence.

SEQ ID NO: 4 sets forth FgenI model RNA synthesized RNA sequence, with primer annealing sites.

SEQ ID NO: 5 sets forth DNA template of FgenI model RNA, with primer annealing sites.

SEQ ID NO: 6 sets forth Chi18-4 model RNA synthesized RNA sequence.

SEQ ID NO: 7 sets forth Chi18-4 model RNA synthesized RNA sequence, primer annealing sites in bold.

SEQ ID NO: 8 sets forth DNA transcript for Chi18-4 model RNA synthesized RNA sequence, primer annealing sites in bold.

SEQ ID NO: 9 sets forth RNA used in the FP Stabilization Assay.

### DETAILED DESCRIPTION OF THE INVENTION

An objective of this invention is to provide a method for preparing a formulation comprising a specific ratio of stabilizer to pharmaceutical substance, including a formulation further comprising one or more lipid nanoparticles (LNP), which maintains product attributes such as colloidal stability and encapsulation. A second objective of this invention is to provide lyophilization processes, in particular for such formulations, to achieve long term stability. A third objective of this invention is to provide methods for preparing stable formulations that maintain product attributes such as colloidal stability and encapsulation during and post-reconstitution and resuspension of a lyophilized mixture, including a lipid nanoparticle formulation, using suitable diluents.

The present invention provides a first method for producing a stable liquid formulation comprising a mixture of a pharmaceutical substance and a stabilizing agent, wherein the method comprises mixing the pharmaceutical substance and the stabilizing agent in a specific ratio, so as to thereby produce the stable formulation.

The present invention provides a second method for lyophilizing a liquid formulation, wherein the method comprises the following steps:
a) providing a liquid formulation comprising a pharmaceutical substance and a stabilizing agent, wherein, in one embodiment, the liquid formulation comprises a specific ratio of the pharmaceutical substance to the stabilizing agent;
b) initiating a freezing step in order to obtain a frozen liquid by:
   i) introducing the liquid formulation provided in step (a) into a freeze drying chamber of a freeze dryer; and
   ii) cooling the liquid formulation to a freezing temperature, wherein the cooling is performed at a defined cooling rate in order to obtain a frozen liquid;
c) initiating a primary drying step of the frozen liquid obtained in step (b) in order to obtain a partly dried product by:
   i) reducing the pressure in the freeze drying chamber to a pressure below the vapor pressure of ice, and
   ii) increasing the shelf temperature;
d) initiating a secondary drying step of the partly dried product obtained in step (c) comprising heating the partly dried product obtained in step (c) to a drying temperature wherein the heating is performed at a defined heating rate in order to obtain a lyophilized composition comprising the pharmaceutical substance and the stabilizing agent; and
e) equilibrating the pressure in the freeze drying chamber to atmospheric pressure and removing the lyophilized composition comprising the pharmaceutical substance and the stabilizing agent obtained in step (d) from the freeze-drying chamber. As used herein “frozen liquid” shall mean a composition comprising ice and a freeze concentrate, wherein the freeze concentrate comprises all materials in the frozen liquid except most of the ice.

In one embodiment, the second method for lyophilizing a liquid formulation comprises the following steps:
a) providing a liquid formulation comprising a pharmaceutical substance and a stabilizing agent, wherein the liquid formulation comprises a specific ratio of the pharmaceutical substance to the stabilizing agent;
b) initiating a freezing step in order to obtain a frozen liquid by:
   i) introducing the liquid formulation provided in step (a) into a freeze drying chamber of a freeze dryer; and
   ii) cooling the liquid formulation to a freezing temperature, wherein the cooling is performed at a defined cooling rate in order to obtain a frozen liquid;
c) initiating a primary drying step of the frozen liquid obtained in step (b) in order to obtain a partly dried product by:
   i) reducing the pressure in the freeze drying chamber to a pressure below the vapor pressure of ice, and
   ii) increasing the shelf temperature;
d) initiating a secondary drying step of the partly dried product obtained in step (c) comprising heating the partly dried product obtained in step (c) to a drying temperature wherein the heating is performed at a defined heating rate in order to obtain a lyophilized composition comprising the pharmaceutical substance and the stabilizing agent; and
e) equilibrating the pressure in the freeze drying chamber to atmospheric pressure and removing the lyophilized composition comprising the pharmaceutical substance and the stabilizing agent obtained in step (d) from the freeze-drying chamber. As used herein “frozen liquid” shall mean a composition comprising ice and a freeze concentrate, wherein the freeze concentrate comprises all materials in the frozen liquid except most of the ice.

In one embodiment of the above methods, the liquid formulation further comprises at least one encapsulating agent. In one embodiment, the encapsulating agent is selected from the group consisting of a lipid, a lipid nanoparticle (LNP), lipoplexes, polymeric particles, polyplexes, and monolithic delivery systems, and a combination thereof. In a preferred embodiment, the encapsulating agent is a lipid nanoparticle (LNP).

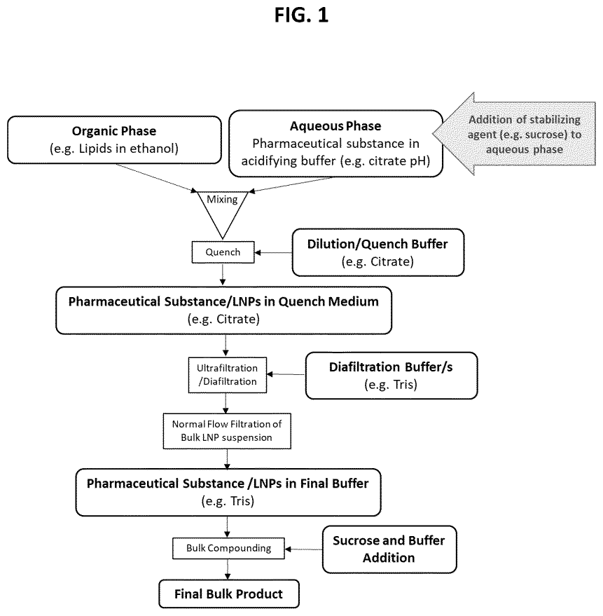

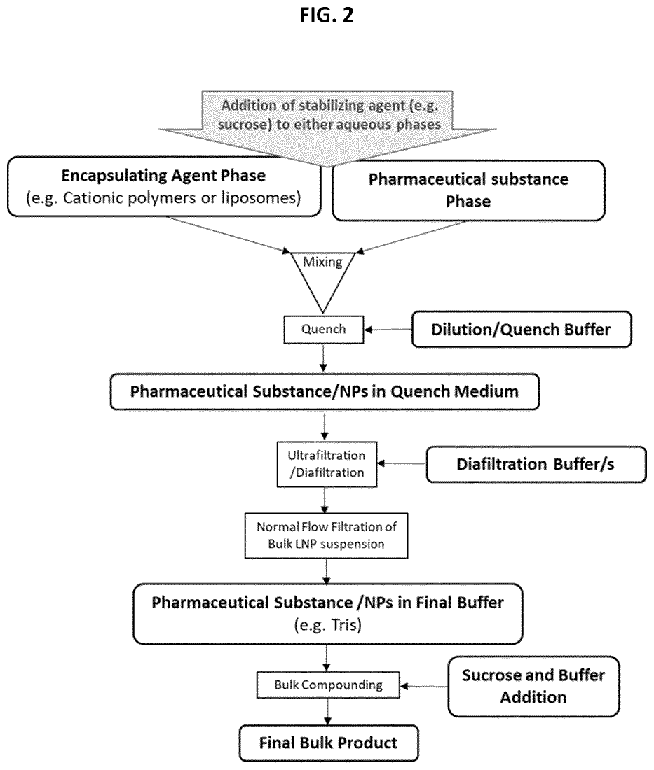

In another embodiment of the above methods, the liquid formulation further comprises a buffer. In some embodiments, suitable liquid formulations contain buffering agents such as tris, histidine, citrate, acetate, phosphate and succinate. The pH of a liquid formulation relates to the pKa of the encapsulating agent (e.g. cationic lipid). As shown in FIGS. 1 and 2, (i) the pH of the acidifying buffer should be at least half a pH scale less than the pKa of the encapsulating agent (e.g. cationic lipid), and (ii) the pH of the final buffer should be at least half a pH scale greater than the pKa of the encapsulating agent (e.g. cationic lipid).

In another embodiment of the above methods, the liquid formulation further comprises a salt. In one embodiment, the salt is a sodium salt. In a preferred embodiment, the salt is NaCl.

In another embodiment of the above methods, the liquid formulation further comprises a surfactant, a preservative, any other excipient, or a combination thereof. As used herein, “any other excipient” includes, but is not limited to, antioxidants, glutathione, EDTA, methionine, desferal, antioxidants, metal scavengers, or free radical scavengers. In one aspect, the surfactant, preservative, excipient or combination thereof is selected from sterile water for injection (sWFI), bacteriostatic water for injection (BWFI), saline, dextrose solution, polysorbates, poloxamers, Triton, divalent cations, Ringer's lactate, amino acids, sugars, polyols, polymers or cyclodextrins.

In another embodiment of the above methods, the pharmaceutical substance is selected from the group consisting of a protein, a peptide, a polysaccharide, a small molecule, a natural product, a nucleic acid, an immunogen, a vaccine, a polymer, a chemical compound, and a combination thereof. In one preferred embodiment, the pharmaceutical substance is a nucleic acid. In another preferred embodiment, the nucleic acid is selected from the group consisting of DNA, RNA, RNA/DNA hybrids, and aptamers. In another preferred embodiment, the RNA is mRNA. In a second preferred embodiment, the pharmaceutical substance is a protein. In another preferred embodiment, the protein is selected from the group consisting of an antibody or a fragment thereof, a growth factor, a clotting factor, a cytokine, a fusion protein, an enzyme, a carrier protein, a polysaccharide-containing antigen, and a combination thereof. In a further preferred embodiment, the antibody is a monoclonal antibody or a single-domain antibody.

In another embodiment of the above methods, the liquid formulation contains various pharmaceutical substance concentrations. In one embodiment, the pharmaceutical substance is at a concentration of <1 mg/ml. In another embodiment, the pharmaceutical substance is at a concentration of at least about 0.05 mg/ml. In another embodiment, the pharmaceutical substance is at a concentration of at least about 0.5 mg/ml. In another embodiment, the pharmaceutical substance is at a concentration of at least about 1 mg/ml. In another embodiment, the pharmaceutical substance concentration is from about 0.05 mg/ml to about 0.5 mg/ml. In another embodiment, the pharmaceutical substance is at a concentration of at least 10 mg/ml. In another embodiment, the pharmaceutical substance is at a concentration of at least 50 mg/ml. In some embodiments, the present invention is particularly useful to prepare liquid formulations containing a pharmaceutical substance at high concentrations. For example, liquid formulations suitable for the present invention may contain a pharmaceutical substance of interest at a concentration of 75 mg/ml, at least about 100 mg/ml, at least about 150 mg/ml, at least about 200 mg/ml, at least about 250 mg/ml, at least about 300 mg/ml, or at least about 400 mg/ml.

In a further embodiment of the above methods, the stabilizing agent is selected from the group consisting of sucrose, mannose, sorbitol, raffinose, trehalose, mannitol, inositol, sodium chloride, arginine, lactose, hydroxyethyl starch, dextran, polyvinylpyrolidone, glycine, and a combination thereof. In a preferred embodiment, the stabilizing agent is sucrose. In another preferred embodiment, the stabilizing agent is trehalose. In a further preferred embodiment, the stabilizing agent is a combination of sucrose and trehalose. In one embodiment, the stabilizing agent concentration includes, but is not limited to, a concentration from about 10 mg/ml to about 400 mg/ml, from about 100 mg/ml to about 200 mg/ml, 103 mg/ml to about 200 mg/ml, or any concentration set forth in Table 1.

In some embodiments, the stabilizing agent (e.g., sucrose, trehalose, or a combination of sucrose and trehalose) concentration includes, but is not limited to a concentration from about 1% w/v to about 20% w/v, from about 5% w/v to about 15% w/v, from about 5% w/v to about 10% w/v, about 5% w/v, about 10% w/v, or any concentration as set forth in Table 8. In a further embodiment, the stabilizing agent is a combination of sucrose and trehalose, present at about equal % w/v.

In a further embodiment of the above methods, the mass amount of the stabilizing agent and the mass amount of the pharmaceutical substance are in a specific ratio. In one embodiment, the ratio of the mass amount of the stabilizing agent and the pharmaceutical substance is no greater than 5000. In another embodiment, the ratio of the mass amount of the stabilizing agent and the pharmaceutical substance is no greater than 2000. In another embodiment, the ratio of the mass amount of the stabilizing agent and the pharmaceutical substance is no greater than 1000. In another embodiment, the ratio of the mass amount of the stabilizing agent and the pharmaceutical substance is no greater than 500. In another embodiment, the ratio of the mass amount of the stabilizing agent and the pharmaceutical substance is no greater than 100. In another embodiment, the ratio of the mass amount of the stabilizing agent and the pharmaceutical substance is no greater than 50. In another embodiment, the ratio of the mass amount of the stabilizing agent and the pharmaceutical substance is no greater than 10. In another embodiment, the ratio of the mass amount of the stabilizing agent and the pharmaceutical substance is no greater than 1. In another embodiment, the ratio of the mass amount of the stabilizing agent and the pharmaceutical substance is no greater than 0.5. In another embodiment, the ratio of the mass amount of the stabilizing agent and the pharmaceutical substance is no greater than 0.1.

In another embodiment of the above methods, the stabilizing agent and pharmaceutical substance comprise a mass ratio of about 200-2000 of the stabilizing agent: 1 of the pharmaceutical substance. In a further embodiment, the pharmaceutical substance is mRNA and the stabilizing agent is sucrose.

In another embodiment of the above methods, the liquid formulation is produced by a method comprising an Alternate Fabrication Processes as set forth in FIG. 1 and FIG. 2.

In another embodiment of the second method, the cooling rate of the freezing step set forth in step (b) is from 0.02° C./min to 37° C./min. In a preferred embodiment, the cooling rate is 0.2° C./min or 0.5° C./min. In another embodiment, the shelf temperature during initial freezing set forth in step (b) is from about −30° C. to −60° C.

In another embodiment of the second method, the cooling rate of the primary drying step set forth in step (c) is selected from (i) a precooled shelf (PCS) at approximately 40° C./min, (ii) 1° C./min, or (iii) 0.5° C./min.

In another embodiment of the second method, the heating rate of the secondary drying step set forth in step (d) is from 0.05° C./min to 1° C./min. In a preferred embodiment, the heating rate is 0.2° C./min.

In a further embodiment of the second method, the shelf temperature during primary drying set forth in step (c) is from about −15° C. to about −30° C. In a preferred embodiment, the shelf temperature is −25° C.

In a further embodiment of the second method, the chamber pressure during primary drying set forth in step (c) is from about 25 mTorr to about 100 mTorr. In a preferred embodiment, the chamber pressure is 50 mTorr.

In another embodiment of the second method, the method further comprises an annealing step after the initial freezing set forth in step (b). “Annealing” is a thermal treatment process, useful for amorphous substances that form a metastable glass with incomplete crystallization when first frozen. During annealing, the product temperature is cycled (for example: from −40° C. to −20° C. for a few hours and then back to −40° C.) to obtain more complete crystallization. Annealing has the added advantage of larger crystal growth and corresponding shorter drying times. In one embodiment of the second method, the annealing temperature is from about −5° C. to about −25° C. In a preferred embodiment, the annealing temperature is −10° C.

In another embodiment of the second method, the lyophilized product comprises amorphous materials. In a further embodiment, the liquid formulation remains amorphous in a freeze concentrate upon freezing, and the freezing temperature set forth in step (b) is set below the glass transition temperature (Tg′) of the freeze concentrate. The term “freeze concentrate” shall mean all materials in the frozen liquid formulation except most of the ice.

In another embodiment of the second method, the liquid formulation is partially crystalline upon freezing, and the freezing temperature set forth in step (b) is set below the eutectic melting temperature (T_{eutectic}) or a secondary melting temperature of the frozen solution. In frozen aqueous multi-component solutions, the term “secondary melting” implies the simultaneous melting of solute crystallized during freezing and/or annealing and the ice phase as defined by the supplemented phase diagram (state diagram. The term is analogous to eutectic melting in the case of a frozen aqueous binary solution where both ice and solute crystallize. Primary melting refers to melting of the ice phase only as defined by a supplemented phase diagram. The term is analogous to ice melting in the case of a frozen aqueous binary solution where both ice and solute crystallize. (Shalaev E Y, Franks F. 2002. Solid-liquid state diagrams in pharmaceutical lyophilisation: Crystallisation of solutes. In: Levine H, editor. Amorphous food and pharmaceutical systems. Cambridge: Royal Society of Chemistry. pp 200-215). In one embodiment of the above methods, the lyophilized product comprises amorphous materials. In another embodiment, the lyophilized product comprises partly crystalline/partly amorphous materials.

The present invention provides a method of improving the stability of a lyophilized pharmaceutical substance or the efficiency of the lyophilization cycle, the method comprising lyophilizing the pharmaceutical substance in a liquid formulation according to the above methods.

The present invention also provides a method of improving the stability of the pharmaceutical substance of the above methods by depressing crystallization of the formulation components.

In a further embodiment of the above methods, the formulation is stored at ambient temperature or subambient temperature for a defined period. As used herein, the term “ambient” shall mean room temperature or a temperature between 15° C.-30° C. Additionally, the term “subambient” as used herein shall mean a temperature below ambient temperature, which includes temperatures below, at or above Tg′ of a frozen formulation.

In a further embodiment of the above methods, the formulation is stable as a frozen matrix, a refrigerated liquid, or a refrigerated lyophilized product. In another embodiment, the formulation is above the glass transition temperature of the frozen matrix (Tg′). In another embodiment, the formulation remains amorphous during storage above Tg′. In another embodiment, the storage temperature is ≤−20° C.

The present invention also provides a method of improving the stability of the lyophilized composition produced by the second method comprising the addition of glutathione, EDTA, methionine, desferal and any antioxidants or metal scavengers to the liquid formulation prior to lyophilization or during reconstitution of the lyophilized composition in a form suitable for injection.

The present invention also provides a lyophilized composition produced using the above methods. Lyophilized products are extremely hygroscopic and they must be sealed in air tight containers (e.g. glass vials) following freeze drying to prevent rehydration from atmospheric exposure. In one embodiment, the lyophilized composition has a cake height of up to 3 cm. In another embodiment, the lyophilized composition has a cake height of from about 0.01 cm to about 3 cm. In another embodiment, the lyophilized composition has a cake height of from about 0.01 cm to about 2.5 cm. In another embodiment, the lyophilized composition has a cake height of from about 0.01 cm to about 2.2 cm. In further embodiment, the lyophilized composition has a cake height of about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3 cm. In a further embodiment, the lyophilized composition has a cake height of <0.01 cm.

The present invention provides a fourth method for preparing a stable formulation comprising the steps of:
(a) lyophilizing a mixture of a pharmaceutical substance and a stabilizing amount of a stabilizing agent which prevents or reduces chemical or physical instability of the pharmaceutical substance upon lyophilization and subsequent storage as set forth in the second method, and
(b) reconstituting the lyophilized mixture of step (a) in a diluent such that the reconstituted formulation is stable.

In one embodiment of the fourth method, the diluent is selected from sterile water for injection (sWFI), saline, dextrose solution, polysorbates, poloxamers, Triton, divalent cations, Ringer's lactate, amino acids, sugars, polyols, polymers or cyclodextrins, pH buffered diluents, or preservative containing diluents such as bacteriostatic water for injection (BWFI), 2-phenoxyethanol, m-cresol, or phenol.

The present invention provides a fifth method of storing a pharmaceutical substance comprising the steps of: (a) producing a stable formulation according to the first method or a lyophilized composition of the second method; and (b) storing the stable formulation or lyophilized composition for a defined period.

The present invention provides a third method of storing a pharmaceutical substance comprising the steps of:
(a) lyophilizing the pharmaceutical substance in a liquid formulation according to any of the above methods comprising a primary drying step executed at a product temperature at, below, or above the collapse temperature; and
(b) storing the lyophilized pharmaceutical substance for a defined period.

In one embodiment of the above methods, the period is longer than 3 months. In another embodiment, the period is longer than 8 months. In another embodiment, the period is longer than 12 months. In another embodiment, the period is longer than 18 months. In a further embodiment, the period is longer than 24 months.

The present invention provides a stable formulation produced using the first method.

The present invention also provides a lyophilized composition produced using the second method.

The present invention also provides the following embodiments:
Embodiment 1. A method for lyophilizing a liquid formulation, wherein the method comprises the following steps:
   a) providing a liquid formulation comprising a pharmaceutical substance and a stabilizing agent;
   b) initiating a freezing step in order to obtain a frozen liquid by:
      i) introducing the liquid formulation provided in step (a) into a freeze drying chamber of a freeze dryer; and
      ii) cooling the liquid formulation to a freezing temperature, wherein the cooling is performed at a defined cooling rate in order to obtain a frozen liquid;
   c) initiating a primary drying step of the frozen liquid obtained in step (b) in order to obtain a partly dried product by:
      i) reducing the pressure in the freeze drying chamber to a pressure below the vapor pressure of ice, and
      ii) increasing the shelf temperature;
   d) initiating a secondary drying step of the partly dried product obtained in step (c) comprising heating the partly dried product obtained in step (c) to a drying temperature wherein the heating is performed at a defined heating rate in order to obtain a lyophilized composition comprising the pharmaceutical substance and the stabilizing agent; and
   e) equilibrating the pressure in the freeze drying chamber to atmospheric pressure and removing the lyophilized composition comprising the pharmaceutical substance and the stabilizing agent obtained in step (d) from the freeze-drying chamber.
Embodiment 2. The method of embodiment 1, wherein the liquid formulation further comprises at least one encapsulating agent.
Embodiment 3. The method of any one of embodiments 1 and 2, wherein the liquid formulation further comprises a buffer.
Embodiment 4. The method of any one of embodiments 1-3, wherein the liquid formulation further comprises a salt.
Embodiment 5. The method of any one of embodiments 1-4, wherein the liquid formulation further comprises a surfactant, a preservative, any other excipient, or a combination thereof.
Embodiment 6. The method of any one of embodiments 1-5, wherein the cooling rate of the freezing step set forth in step (b) is from 0.02° C./min to 37° C./min.
Embodiment 7. The method of embodiment 6, wherein the cooling rate is 0.2° C./min or 0.5° C./min.
Embodiment 8. The method of any one of embodiments 1-7, wherein the cooling rate of the primary drying step set forth in step (c) is selected from
   (i) a precooled shelf (PCS) at approximately 4° C./min,
   (ii) VC/min, or
   (iii) 0.5° C./min.
Embodiment 9. The method of any one of embodiments 1-8, wherein the heating rate of the secondary drying step set forth in step (d) is from 0.05° C./min to 1° C./min.
Embodiment 10. The method of embodiment 9, where the heating rate is 0.2° C./min.
Embodiment 11. The method of any one of embodiments 1-10, wherein the liquid formulation is prepared using Tangential Flow Filtration (TFF).
Embodiment 12. The method of any one of embodiments 1-11, wherein the liquid formulation is produced by a method comprising an Alternate Fabrication Processes as set forth in FIG. 1 and FIG. 2.
Embodiment 13. The method of any one of embodiments 1-12, further comprising an annealing step after the initial freezing set forth in step (b).
Embodiment 14. The method of any one of embodiments 1-13, wherein the liquid formulation remains amorphous in a freeze concentrate upon freezing, and the freezing temperature set forth in step (b) is set below the glass transition temperature (Tg′) of the freeze concentrate.
Embodiment 15. The method of any one of embodiments 1-13, wherein the liquid formulation is partially crystalline upon freezing, and the freezing temperature set forth in step (b) is set below the eutectic melting temperature (T_{eutectic}) or a secondary melting temperature of the frozen solution.
Embodiment 16. The method of any one of embodiments 1-15, wherein the ratio of the mass amount of the stabilizing agent and the pharmaceutical substance is no greater than 1000.
Embodiment 17. The method of any one of embodiments 1-15, wherein the ratio of the mass amount of the stabilizing agent and the pharmaceutical substance is no greater than 500.
Embodiment 18. The method of any one of embodiments 1-15, wherein the ratio of the mass amount of the stabilizing agent and the pharmaceutical substance is no greater than 100.
Embodiment 19. The method of any one of embodiments 1-15, wherein the ratio of the mass amount of the stabilizing agent and the pharmaceutical substance is no greater than 50.
Embodiment 20. The method of any one of embodiments 1-15, wherein the ratio of the mass amount of the stabilizing agent and the pharmaceutical substance is no greater than 10.
Embodiment 21. The method of any one of embodiments 1-15, wherein the ratio of the mass amount of the stabilizing agent and the pharmaceutical substance is no greater than 1.
Embodiment 22. The method of any one of embodiments 1-15, wherein the ratio of the mass amount of the stabilizing agent and the pharmaceutical substance is no greater than 0.5.
Embodiment 23. The method of any one of embodiments 1-15, wherein the ratio of the mass amount of the stabilizing agent and the pharmaceutical substance is no greater than 0.1.
Embodiment 24. The method of any one of embodiments 1-23, wherein the pharmaceutical substance is at a concentration of <1 mg/ml.
Embodiment 25. The method of any one of embodiments 1-23, wherein the pharmaceutical substance is at a concentration of at least about 1 mg/ml.
Embodiment 26. The method of any one of embodiments 1-23, wherein the pharmaceutical substance is at a concentration of at least 10 mg/ml.
Embodiment 27. The method of any one of embodiments 1-23, wherein the pharmaceutical substance is at a concentration of at least 50 mg/ml.
Embodiment 28. The method of any one of embodiments 1-27, wherein the stabilizing agent is selected from the group consisting of sucrose, mannose, sorbitol, raffinose, trehalose, mannitol, inositol, sodium chloride, arginine, lactose, hydroxyethyl starch, dextran, polyvinylpyrolidone, glycine, and a combination thereof.
Embodiment 29. The method of embodiment 27, wherein the stabilizing agent is sucrose.
Embodiment 30. The method of embodiment 27, wherein the stabilizing agent is trehalose.
Embodiment 31. The method of embodiment 27, wherein the stabilizing agent is a combination of sucrose and trehalose.
Embodiment 32. The method of any one of embodiments 1-31, wherein the lyophilized product comprises amorphous materials.
Embodiment 33. The method of any one of embodiments 1-31, wherein the lyophilized product comprises partly crystalline/partly amorphous materials.
Embodiment 34. The method of any one of embodiments 1-33, wherein the pharmaceutical substance is selected from the group consisting of a protein, a peptide, a polysaccharide, a small molecule, a natural product, a nucleic acid, an immunogen, a vaccine, a polymer, a chemical compound, and a combination thereof.
Embodiment 35. The method of embodiment 34, wherein the pharmaceutical substance is a nucleic acid.
Embodiment 36. The method of embodiment 35, wherein the nucleic acid is selected from the group consisting of DNA, RNA, RNA/DNA hybrids, and aptamers.
Embodiment 37. The method of embodiment 36, wherein the RNA is mRNA.
Embodiment 38. The method of embodiment 34, wherein the pharmaceutical substance is a protein.
Embodiment 39. The method of embodiment 38, wherein the protein is selected from the group consisting of an antibody or a fragment thereof, a growth factor, a clotting factor, a cytokine, a fusion protein, an enzyme, a carrier protein, a polysaccharide-containing antigen, and a combination thereof.
Embodiment 40. The method of embodiment 39, wherein the antibody is a monoclonal antibody or a single-domain antibody.
Embodiment 41. The method of any one of embodiments 2-40, wherein the encapsulating agent is selected from the group consisting of a lipid, a lipid nanoparticle (LNP), lipoplexes, polymeric particles, polyplexes, and monolithic delivery systems, and a combination thereof.
Embodiment 42. The method of embodiment 41, wherein the encapsulating agent is a lipid nanoparticle (LNP).
Embodiment 43. A lyophilized composition produced using the method of any one of embodiments 1-42.
Embodiment 44 A method of storing a pharmaceutical substance comprising the steps of:
   (a) lyophilizing the pharmaceutical substance in a liquid formulation according to the method of any one of embodiments 1-42 comprising a primary drying step executed at a product temperature at, below, or above the collapse temperature; and
   (b) storing the lyophilized pharmaceutical substance for a defined period.
Embodiment 45. The method of embodiment 44, wherein the period is longer than 3 months.
Embodiment 46. The method of embodiment 44, wherein the period is longer than 8 months.
Embodiment 47. The method of embodiment 44, wherein the period is longer than 12 months.
Embodiment 48. The method of embodiment 44, wherein the period is longer than 18 months.
Embodiment 49. The method of embodiment 44, wherein the period is longer than 24 months.
Embodiment 50. A method of improving the stability of a lyophilized pharmaceutical substance or the efficiency of the lyophilization cycle, the method comprising lyophilizing the pharmaceutical substance in a liquid formulation according to the methods of any one of embodiments 1-42.
Embodiment 51. A method of improving the stability of the pharmaceutical substance of embodiment 1 by depressing crystallization of the formulation components.
Embodiment 52. A method of improving the stability of the lyophilized composition of embodiment 1 by adding glutathione, EDTA, methionine, desferal and any antioxidants or metal scavengers to the liquid formulation prior to lyophilization or during reconstitution of the lyophilized composition in a form suitable for injection.

### A. General Lyophilization and Cycle Optimization

Lyophilization includes the sequential steps of freezing, primary drying, and secondary drying. The primary drying step, the longest and therefore most expensive step of the lyophilization process, is very sensitive to deviations in process parameters, including the process parameters of shelf temperature and chamber pressure.

Current lyophilization methods for biological and pharmaceutical substances maintain a constant shelf temperature and a constant chamber pressure throughout the primary drying step, which simplifies the primary drying step of the lyophilization process. However, constant process parameters of shelf temperature and chamber pressure throughout the duration of the primary drying step decrease the efficiency of the primary drying step and increase the cost of the primary drying step.

It is desirable to decrease the length, and therefore the expense, of the primary drying step. PCT Publication No. WO2008042408, which is incorporated by reference herein in its entirety, discloses general lyophilization and cycle optimization methods that are useful in the present invention and are described herein. According to various embodiments set forth therein, the length of the primary drying step is decreased by modifying the process parameters of shelf temperature and chamber pressure to maintain the product temperature of the pharmaceutical substance at or just below the target temperature of the pharmaceutical substance throughout the primary drying step. The product temperature of a pharmaceutical substance is the temperature of the pharmaceutical substance at any given time point during lyophilization. When measured in-time using a pilot-scale lyophilizer or a laboratory-scale lyophilizer, the product temperature of a pharmaceutical substance is often measured at a position within the pharmaceutical substance at the bottom of the vial. The target temperature of a pharmaceutical substance is the desired temperature of the pharmaceutical substance at any given time point during lyophilization and is typically about 2-3° C. below the collapse temperature of the pharmaceutical substance. The collapse temperature of a pharmaceutical substance is the temperature during freezing resulting in the collapse of the structural integrity of the pharmaceutical substance.

The relationship between heat and mass balance during the primary drying step are described by the following equation:

$${( \frac{\partial m}{\partial t} )}_{i} = \frac{{S}_{i n} * {( {P}_{S u b l} - {P}_{Chamber} )}_{i}}{{R ( h )}_{i}} = \frac{{S}_{out} * {K}_{V} ( P ) * {( {T}_{Shelf} - {T}_{product} )}_{i}}{Δ {H}_{s}} \qquad Equation 1$$

where

$${( \frac{\partial m}{\partial t} )}_{i} = sublimation rate ,$$

Kᵥ=vial heat transfer coefficient,
T_{shelf}=shelf temperature (typically inlet temperature of heat transfer liquid),
T_{product}=product temperature (typically measured just above the vial bottom),
ΔHₛ=specific heat of sublimation,
Sₒᵤₜ=external surface area of vial,
Sᵢₙ=internal surface area of vial,
P_{subl}=pressure of water vapor over sublimation surface,
P_{chamber}=chamber pressure, and
R(h)ₗ=dry cake resistance at dry layer height (h)ₗ.

During the primary drying step, the specific heat of sublimation (ΔHₛ), the external surface of the vial (Sₒᵤₜ), the internal surface of the vial (Sᵢₙ), and the vial heat transfer coefficient (Kᵥ) remain relatively constant. However, as water is removed from the pharmaceutical substance and as the sublimation front moves gradually from the top of the vial to the bottom of the vial, the total cake resistance gradually increases due to the development of a dry layer within the material.

Cake resistance is the resistance of dry porous material to the flow of water vapor generated during sublimation. In general, cake resistance depends on the concentration of solids in the material and the nature of the material undergoing lyophilization. Cake resistance increases as the concentration of solids in the material increases.

However, the solids concentration is not the only factor affecting cake resistance. Materials subject to lyophilization, including, for example, biological agents (e.g., proteins, peptides and nucleic acids) and pharmaceutical agents (e.g., small molecules), often include bulking agents, stabilizers, buffers and other product formulation components in addition to a solvent. Exemplary bulking agents include sucrose, glycine, sodium chloride, lactose and mannitol. Exemplary stabilizers include sucrose, trehalose, arginine and sorbitol. Exemplary buffers include tris, histidine, citrate, acetate, phosphate and succinate. Exemplary additional formulation components include antioxidants, metal scavengers, surface active agents and tonicity components. Formulation components can affect the cake resistance of a material and, therefore, the process parameters necessary to efficiently lyophilize a selected material. Exemplary solvents include water, organic solvents and inorganic solvents. An exemplary material, a 5% sucrose solution, has a lower relative cake resistance than a mannitol-sucrose buffer having the same solids concentration. Sucrose is susceptible to partial collapse at temperatures close to −32° C., resulting in the formation of larger pores and, therefore, less resistance to water vapor flow. This may account for the relatively small cake resistance of a 5% sucrose solution as compared to a mannitol-based formulation. As a result, the product temperature of a 5% sucrose solution does not increase more than 5° C. during the primary drying step of lyophilization.

In the case of the exemplary 5° C. increase in product temperature, the increased complexity of modifying the shelf temperature and/or the chamber pressure of the lyophilizer may outweigh the benefits of decreasing the duration of the primary drying step. Therefore, the process parameters of constant shelf temperature and constant chamber pressure are reasonable for this material. When drying time is critical, adjustment of the shelf temperature and the pressure to optimize duration of cycle is possible.

In practice, a 5° C. increase in product temperature during the primary drying step of lyophilization is exemplary of a reasonable rise in temperature. Therefore, in the case of a 5% sucrose solution, for example, it is not necessary to change the shelf temperature and/or chamber pressure process parameters during the primary drying step of lyophilization. Similarly, it is not necessary to change the shelf temperature and/or chamber pressure process parameters during the primary drying stage of similar materials with similarly low pharmaceutical substance concentration and relatively small, for example less than 5%, solids concentration.

However, as the solids concentration in a material increases, for example, as the pharmaceutical substance concentration increases, the cake resistance of the material also increases. A higher solids concentration also results in a greater increase in product temperature during a primary drying step wherein the shelf temperature and the chamber pressure remain constant.

According to the exemplary primary drying step of a higher protein concentration material (result not shown), the product temperature of the material increased from −40° C. to −18° C. The exemplary 22° C. increase in product temperature is considered rather large and economically unacceptable. Moreover, the product temperature of the material increased above its target temperature of −20° C. Therefore, maintaining the chosen process parameters at constant values is considered economically unacceptable for this high protein concentration material.

The product temperature of the exemplary higher protein concentration material can be maintained below the target temperature of −20° C. during the primary drying step of lyophilization by resetting the shelf temperature and/or the chamber pressure process parameters to constant, but relatively lower, values. Constant process parameters of shelf temperature and chamber pressure can be calculated using Equation 1 such that the product temperature never exceeds the target temperature at the end of the primary drying step. Although selecting a constant shelf temperature and a constant chamber pressure for lyophilization of higher protein concentration materials or higher cake resistance materials is a safe and simple solution from a manufacturing perspective, this method results in a very long and therefore very expensive primary drying step.

Analysis of Equation 1 suggests, however, that maintaining a constant shelf temperature and a constant chamber pressure is not the most economical method of conducting the primary drying step for higher protein concentration materials or higher cake resistance materials. Alternatively, either and/or both of the process parameters of shelf temperature and chamber pressure can be modified during the course of the primary drying step to maintain an optimal product temperature of a material during the primary drying step.

A mathematical model can be constructed based on Equation 1. An exemplary mathematical model describes the relationship between the process parameters of chamber pressure and shelf temperature, the dry product cake resistance, the vial heat transfer coefficient, and the product temperature. The mathematical model can be utilized to calculate a product temperature profile for a selected material. First, the mathematical model can be used to estimate the product temperature of a specific material with known product properties at each time point measurement of the process parameters during the primary drying step. Following estimation of the product temperature, the sublimation rate at each time point of the primary drying step can be calculated using the mathematical model and plotted as a function of time. The total sublimated mass of water at each point of the process can be estimated by integrating the sublimation rate profile until the calculated value of sublimated water reaches the total water content of the material. The optimal product temperature profile can be maintained throughout the course of the primary drying step for a specific material by manipulating the process parameters of shelf temperature and/or chamber pressure during the primary drying step.

According to a preferred embodiment, the mathematical model based on Equation 1 described above is used to calculate a product temperature profile for a selected material. Any mathematical model which sufficiently describes the product temperature profile during the primary drying step can be used to generate the designed primary drying cycle. A preferred mathematical model calculates a product temperature profile within 1° C. of the actual product temperature and at or within 2° C. below the target temperature of the material during the course of the primary drying step.

The product temperature profile obtained in the laboratory, pilot or commercial primary drying cycle is used to generate a designed primary drying cycle (based on calculated cake resistance and vial heat transfer coefficients) wherein the product temperature of the material is maintained at a substantially constant temperature and at or just below the target temperature of the selected material during the course of the primary drying step. According to a preferred embodiment, the designed primary drying cycle maintains the product temperature of the material within about 1° C. of the target temperature during the course of the primary drying step. According to another embodiment, the designed primary drying cycle maintains the product temperature of a material with a low collapse temperature, for example, a collapse temperature of about −30° C., within about 5° C. of the target temperature. An exemplary material with a low collapse temperature is sucrose. According to another embodiment, the designed primary drying cycle maintains the product temperature of a material with a relatively higher collapse temperature, for example, a collapse temperature of about −5° C. to −20° C., within about 15° C. of the target temperature.

The target product temperature is also described as the critical temperature of the material, a temperature normally about 2-3° C. below the collapse temperature of the material. The critical temperature of a material is the temperature above which material degrades much more quickly as compared to normal temperature (blow critical). Depending on the material, the critical temperature of a material can be the same as the collapse temperature of the material. Maintaining the material at or just below the target temperature of the material results in the shortest and most efficient primary drying step.

According to one embodiment, the product temperature is maintained at or just below the target temperature of the material by first increasing the shelf temperature to the maximum allowed temperature of the lyophilizer. According to one exemplary embodiment, the maximum allowed temperature of the lyophilizer is in the range of about −30° C. to 60° C., more preferably about 0° C. to 60° C., and most preferably about 20° C. to 60° C.

At the initiation of the primary drying step, cake resistance is not a significant factor in the efficiency of the primary drying rate or sublimation rate; the product temperature is relatively low; and the product temperature depends, for the most part, on chamber pressure. As water is removed from the material, product dry layer begins to form. When product dry layer begins to form, the product temperature begins to gradually increase until the product temperature reaches the target temperature of the material. At the point when the material reaches its target temperature, either the shelf temperature or the chamber pressure or both process parameters are simultaneously adjusted to maintain the material at a temperature at or just below the target temperature of the material.

Continuing for the remainder of the primary drying step, the shelf temperature and the chamber pressure are monitored and, optionally and when necessary, adjusted or modified to maintain the product temperature at or just below the target temperature of the material. It is understood that the terms adjust or modify, when applied to a process parameter, contemplate increasing the value of the parameter and/or decreasing the value of the parameter.

Due to sterility requirements and the automation of load and unload processes in commercial biological and pharmaceutical material lyophilization facilities, it is not practical yet to introduce in-time product temperature sensors into modern commercial-scale lyophilizers. Therefore, it is not widely acceptable in modern manufacturing to measure product temperature at commercial scale and, in response, modify the shelf temperature and/or chamber pressure to maintain an optimal product temperature profile. However, the mathematical model can be used to calculate and/or to validate a designed primary drying cycle for a specific material. A commercial-scale or pilot-scale lyophilizer then can be programmed according to the designed primary drying cycle to modify the shelf temperature and/or the chamber pressure by a predetermined change in value at one or more predetermined time points in the primary drying cycle to optimize the primary drying step for the selected material.

During the primary drying cycle, three programmed parameters—shelf temperature, chamber pressure and time—yield the resulting product temperature profile. These programmed parameters also affect lyophilizer performance, including the rate of sublimation and the rate and efficiency of heat transfer from the shelf to the vial. The optimal process parameters can be measured and/or calculated using a laboratory-scale lyophilizer with an in-time product temperature sensor to create a designed primary drying cycle for pilot-scale or commercial-scale lyophilization of a selected material.

According to one embodiment, prior to generating in-time process parameter measurements, product properties of the selected material can be defined. Exemplary product properties include product water content, liquid product density, frozen product density, and product cake resistance as a function of dry product height. Vial properties also can be defined. Exemplary vial properties include vial filling volume, vial geometry, and vial heat transfer coefficients as a function of pressure. Lyophilization chamber properties also can be defined. Exemplary lyophilization chamber properties include the heat radiation from the lyophilizer walls or door to the product, also known as edge effect.

Knowing some or all of the above-identified product, vial and/or chamber properties, additional lyophilization process properties can be calculated using equations known to one of skill in the art. Exemplary additional properties that can be calculated include the heat flux through the layer of frozen material at any given time, the total heat flux for sublimation, the sublimation rate for an individual vial, the sublimation rate as a function of the primary drying time, pressure over the sublimation surface, the temperature of the sublimation surface at various time points in the cycle, the amount of sublimated ice at various time points in the cycle, the thickness of the frozen layer at the beginning of primary drying and at various additional time points in the cycle (also described as the cake height), and the total sublimation cycle time.

According to a preferred embodiment, a designed primary drying cycle is created by measuring the process parameters and product properties of a selected material using an in-time product temperature sensor in a laboratory-scale lyophilizer over the course of at least one primary drying cycle followed by optimization of the process parameters according to the mathematical model described in greater detail above. The primary drying cycle is optimized when the product temperature of the material is maintained at or just below, within about 1° C. of, the target temperature of the material during the primary drying step.

Using the mathematical model, an estimation is created of the product temperature profile for the subsequent cycles as a function of the process parameters and product properties throughout the course of the entire primary drying step for the selected material. Using the product temperature profile estimation and known characteristics of the pilot-scale or commercial-scale lyophilizer, including vial heat transfer coefficient and edge effect, a primary drying cycle can be designed for a pilot-scale or commercial-scale lyophilizer for efficiently lyophilizing a selected material.

According to one embodiment, the chamber pressure of a lyophilizer is adjusted to known values of pressure during the course of at least one primary drying cycle and a product temperature profile is created by optimizing an appropriate and optionally adjustable shelf temperature using the mathematical model. According to another embodiment, the shelf temperature of a lyophilizer is adjusted to known values of temperature during the course of at least one primary drying cycle and a product temperature profile is created by optimizing an appropriate and optionally adjustable chamber pressure using the mathematical model. According to a further embodiment, a product temperature profile is created by optimizing an appropriate and optionally adjustable chamber pressure and shelf temperature using the mathematical model wherein only the product properties of the material and the vial are known.

Vial heat transfer coefficients are calculated from the weight loss during sublimation during a short period of time. Vial heat transfer coefficients can be calculated using the following equation:

$${K}_{v} = \frac{2 Δ {{H}_{S} ( {m}_{ice} )}_{vial}}{{S}_{out} {\sum }_{i = 1}^{n} ( Δ {T}_{i} + Δ {T}_{i - 1} ) ( {t}_{i} - {t}_{i - 1} )}$$

where
Kᵥ=heat transfer coefficient from heat transfer fluid to product in vial;
ΔHₛ=heat of ice sublimation;
(m_{ice})ᵥᵢₐₗ=amount of ice in the vial;
Sₒᵤₜ=surface area of the bottom of the vial;
ΔTᵢ=actual temperature gradient between product and shelf at the i time point; and
tᵢ=any given (recorded) time point during sublimation of ice.

According to one exemplary lyophilizer, vial heat transfer coefficients as a function of chamber pressure were measured for three sizes of commonly used tubing vials, both as vials in the center of the pilot-scale lyophilizer and as vials at the edge of the lyophilizer. In all cases in the exemplary trials, the heat transfer coefficients in the commercial-scale pilot lyophilizers were lower than the heat transfer coefficients measured in the laboratory-scale lyophilizers.

An exemplary designed primary drying cycle was created by inputting measured values into the mathematical model based on Equation 1 (see cycles in Examples 1-3). The predicted product temperature profile based on the designed primary drying cycle in the commercial-scale pilot lyophilizer was in agreement with the measured product temperature values during laboratory-scale lyophilization of the same selected material, validating the designed primary drying cycle.

According to one embodiment, the designed primary drying cycle modifies shelf temperature at least once during the course of the primary drying step. According to another embodiment, the designed primary drying cycle modifies chamber pressure at least once during the course of the primary drying step. According to a further embodiment, the designed primary drying cycle modifies each of the shelf temperature and the chamber pressure at least once during the course of the primary drying step.

In another aspect, the invention is a commercial-scale lyophilizer, a pilot-scale lyophilizer, or a laboratory-scale lyophilizer programmed to perform a designed primary drying cycle for a selected material.

According to one embodiment of the programmed lyophilizer, the lyophilizer is programmed to modify the shelf temperature at least once during the primary drying step. According to another embodiment, the lyophilizer is programmed to modify the chamber pressure at least once during the primary drying step. According to a further embodiment, the lyophilizer is programmed to modify each of the shelf temperature and the chamber pressure at least once during the primary drying step.

### B. Lyophilization Above Collapse Temperature

The collapse temperature is the product temperature during freeze-drying above which product cake begins to lose its original structure. It was reported in the literature that, above the collapse temperature, a product could experience slow sporadic bubbling, swelling, foaming, cavitation, fenestration, gross collapse, retraction and beading that may have consequences on the appearance of the product (MacKenzie, “Collapse during freeze-drying-Qualitative and quantitative aspects” In Freeze-Drying and Advanced Food Technology; Goldblith, S. A., Rey. L, Rothmayr, W. W., Eds.; Academic Press, New York, 1974, 277-307). As a result, it is thought that collapse results in poor product stability, long drying times (due to pore's collapse), uneven drying and loss of texture (R. Bellows, et al. “Freeze-drying of aqueous solutions: maximum allowable operating temperature,” Cryobiology, 9, 559-561 (1972).

The present invention provides highly efficient and cost-effective lyophilization methods. Among other things, PCT Publication No. WO2008042408, which is incorporated by reference herein in its entirety, discloses methods of lyophilizing liquid formulations including a primary drying step at a product temperature at or above the collapse temperature.

Embodiments set forth therein, are particularly useful for freeze-drying liquid formulations containing high concentrations pharmaceutical substances and improving the stability of lyophilized products.

Lyophilization, also known as freeze-drying, is often used to store pharmaceutical drug products (i.e. pharmaceutical substances) because chemical and physical degradation rates of the drug products may be significantly reduced in the dried state, allowing for longer product shelf life. However, lyophilization typically adds significantly to the cost of drug manufacturing. This cost can be minimized by developing a cycle that consumes the least amount of time without jeopardizing product quality or stability. For example, increasing product temperature by 1° C. degree during lyophilization could result in 13% decrease of primary drying time. See, Pikal et al. “The collapse temperature in freeze-drying: dependence of measurement methodology and rate of water removal from the glassy phase,” International Journal of Pharmaceutics, 62 (1990), 165-186.

Traditionally, it was considered critical to maintain the product temperature below its collapse temperature during the primary drying in order to keep intact microscopic structure of solid materials present in the frozen solution. It was thought that it is this structure that makes up the freeze-dried cake with a relatively high surface area, allowing low residual moisture and rapid reconstitution after freeze-drying.

However, as described in PCT Publication No. WO2008042408 set forth above, lyophilization, in particular, primary drying, may be executed at a product temperature above the collapse temperature while maintaining product stability and other desirable quality attributes (e.g., residual moisture, reconstitution time, etc.). Even samples with apparent collapse (e.g., visually detectable collapse in vials), which would be normally rejected, exhibited a similar stability profile to the samples lyophilized below the collapse temperature. Moreover, in some cases, the stability of lyophilized products was improved by freeze-drying above the collapse temperature. For example, partly crystalline/partly amorphous materials lyophilized well above the collapse temperature but slightly below the melting point of mannitol showed better stability than samples lyophilized below the collapse temperature. Thus, this method provides significant economic advantages by providing aggressive and/or fast lyophilization cycles with shorter primary drying time without jeopardizing protein quality and stability.

Another advantage of this method is an application to the assessment of deviations during the commercial manufacturing. If deviation of process parameters during existing commercial cycle (normally performed below the collapse temperature) results in visually detectable product collapse, the present inventors contemplate that the stability profile of the collapsed product may be comparable to the normal cycle if the residual moisture is within specification. Therefore, a particular batch containing samples with visually detectable cake collapse could be released. Thus, manufacturing of commercial batches with zero or substantially reduced reject rates is possible if the particular product could withstand the collapse. A development robustness study can be performed prior to commercial manufacturing to confirm if the stability of the collapsed materials is comparable to that of the control materials for each particular product.

As used herein, the term “collapse temperature (Tc)” refers to a temperature (e.g., product temperature) during freeze-drying at or above which the collapse occurs. As used herein, the term “collapse” refers to loss of an intact structure or change of the original structure of lyophilized cake. In some embodiments, collapse includes loss of a microscopic structure (also referred to as micro-collapse). In some embodiments, micro-collapse is visually undetectable. In some embodiments, micro-collapse refers to loss of less than about 1% (e.g., less than about 0.9%, 0.8%, 0.7%, 0.6%, 0.5%, 0.4%, 0.3%, 0.2%, 0.1%, 0.05%, or 0.01%) of the original intact structure (e.g., a lyophilized cake structure). In some embodiments, the temperature at or above which the micro-collapse occurs is referred to as the micro-collapse temperature. In some embodiments, collapse includes loss of gross structures (also referred to as gross collapse or macro-collapse). In some embodiments, the temperature at or above which the gross collapse occurs is referred to as the gross collapse temperature (or macro-collapse temperature). Typically, gross collapse or macro-collapse results in visually detectable collapse in the lyophilized product. As used herein, the terms “gross collapse,” “macro-collapse,” and “visually detectable collapse” are used interchangeably. In some embodiments, gross collapse, macro-collapse or visually detectable collapse refers to loss of at least 0.1% (e.g., at least about 1%, 5%, 10%, 15%, 20%, 25%, 30%, 35%, 40%, 45%, 50%, 55%, 60%, 65%, 70%, 75%, 80%, 85%, 90%, 95%, or 100%) of the original intact structure (e.g., a lyophilized cake structure).

In some embodiments, the temperature at which collapse occurs may not be discrete. Instead, collapse may be a gradual process that takes place over a temperature range with the intact cake structure progressively disappearing over the temperature range. Typically, the initial change or loss of the intact structure during the lyophilization process is considered the onset of the collapse. The temperature at which this initial change was observed is typically referred to as the onset collapse temperature. The temperature at which the loss of the structure or the structure change appeared to be complete throughout the cake is referred to as the collapse complete temperature.

Collapse in the product during lyophilization may be detected by various instruments including, but not limited to, product temperature measurement devices, freeze-drying microscopy or instruments detecting electrical resistance. Collapse in lyophilized product (e.g., cake) may be detected manually by visual inspection, residual moisture, Differential Scanning Calorimetry (DSC), BET surface area.

Collapse phenomenon is sensitive to the nature of the materials involved. For example, sucrose dominated formulations are very sensitive to collapse especially if they also contain small molecular species such as salts and buffers (Shalaev et al. “Thermophysical properties of pharmaceutically compatible buffers at sub-zero temperatures: implications for freeze-drying,” Pharmaceutical Research (2002), 19(2):195-201). In these formulations, collapse usually occurs at temperature close to the mid-point of glass transition. The viscosity of amorphous sucrose-salt-buffer systems is very low resulting in massive collapse of structure when product temperature exceeds this critical temperature during primary drying. Thus, traditionally, lyophilization is carried out under Tg′ whenever possible.

When product concentration increases, it changes the structural resistance of cake to the collapse.

The present invention may be utilized to lyophilize liquid formulations containing various product concentrations. In some embodiments, the present invention is particularly useful to lyophilize liquid formulations containing pharmaceutical substance at high concentrations. For example, liquid formulations suitable for the present invention may contain a pharmaceutical substance of interest at a concentration of at least about 1 mg/ml, at least about 10 mg/ml, at least about 20 mg/ml, at least about 30 mg/ml, at least about 40 mg/ml, at least about 50 mg/ml, at least about 75 mg/ml, at least about 100 mg/ml, at least about 150 mg/ml, at least about 200 mg/ml, at least about 250 mg/ml, at least about 300 mg/ml, at least about 400 mg/ml. In some embodiments, liquid formulations suitable for the present invention may contain a pharmaceutical substance of interest at a concentration less than 1 mg/ml, or in the range of about 1 mg/ml to 400 mg/ml (e.g., about 1 mg/ml to 50 mg/ml, 1 mg/ml to 60 mg/ml, 1 mg/ml to 70 mg/ml, 1 mg/ml to 80 mg/ml, 1 mg/ml to 90 mg/ml, 1 mg/ml to 100 mg/ml, 100 mg/ml to 150 mg/ml, 100 mg/ml to 200 mg/ml, 100 mg/ml to 250 mg/ml, or 100 mg/ml to 300 mg/ml, or 100 mg/ml to 400 mg/ml).

In some embodiments, a suitable formulation contains one or more stabilizing agents (e.g., sucrose, mannose, sorbitol, raffinose, trehalose, glycine, mannitol, sodium chloride, arginine, lactose, hydroxyethyl starch, dextran or polyvinylpyrolidone). In some embodiments, the ratio of the mass amount of the stabilizing agent and the pharmaceutical substance (e.g., nucleic acid) is no greater than 1000 (e.g., no greater than 500, no greater than 250, no greater than 100, no greater than 50, no greater than 10, no greater than 1, no greater than 0.5, no greater than 0.1). In some embodiments, suitable liquid formulations further include one or more bulking agents such as sodium chloride, lactose, mannitol, glycine, sucrose, trehalose and hydroxyethyl starch. In some embodiments, suitable liquid formulations contain buffering agents such as tris, histidine, citrate, acetate, phosphate and succinate. In some embodiments, liquid formulations suitable for the present invention contain amorphous materials. In some embodiments, liquid formulations suitable for the present invention contain a substantial amount of amorphous materials (e.g., sucrose-based formulations). In some embodiments, liquid formulations suitable for the present invention contain partly crystalline/partly amorphous materials.

Lyophilized product in accordance with the present invention can be assessed based on product quality analysis, reconstitution time, quality of reconstitution, high molecular weight, moisture, glass transition temperature, and biological or biochemical activity. Typically, in addition to assays listed in Table 5 product quality analysis includes product degradation rate analysis using methods including, but not limited to, size exclusion HPLC (SE-HPLC), cation exchange-HPLC (CEX-HPLC), X-ray diffraction (XRD), modulated differential scanning calorimetry (mDSC), reversed phase HPLC (RP-HPLC), multi-angle light scattering detector (MALS), fluorescence, ultraviolet absorption, nephelometry, capillary electrophoresis (CE), SDS-PAGE, and combinations thereof. In some embodiments, evaluation of lyophilized product in accordance with the present invention does not include a step of evaluating cake appearance. Additionally, lyophilized product may be assessed based on biological or biochemical activities of the product, typically, after reconstitution.

Inventive methods in accordance with the present invention can be utilized to lyophilize any materials, in particular, pharmaceutical substances. As used herein, the term “pharmaceutical substances” refers to any compounds or entities that alter, inhibit, activate, or otherwise affect biological or chemical events in vivo or in vitro. For example, pharmaceutical substances may include, but are not limited to, proteins, peptides, nucleic acids (e.g., RNAs, DNAs, or RNA/DNA hybrids, aptamers), chemical compounds, polysaccharides, small molecules, drug substances, natural products, immunogens, vaccines, carbohydrates, and/or other products. In some embodiments, the present invention is utilized to lyophilize proteins including, but not limited to, antibodies (e.g., monoclonal antibodies) or fragments thereof, growth factors, clotting factors, cytokines, fusion proteins, polysaccharide antigens, pharmaceutical drug substances, vaccines, enzymes. In some embodiments, the present invention is utilized to lyophilize antibodies or antibody fragments including, but not limited to, intact IgG, F(ab′)2, F(ab)2, Fab′, Fab, ScFv, single domain antibodies (e.g., shark single domain antibodies (e.g., IgNAR or fragments thereof)), diabodies, triabodies, tetrabodies.

In some embodiments, the present invention is used to lyophilize vaccines or vaccine components. Suitable vaccines include, but are not limited to, killed-virus vaccines, attenuated-virus vaccines, toxoid vaccines, subunit vaccines, multi-valent vaccines, conjugate vaccines, live-virus vaccines. Suitable vaccine components include, but are not limited to, polysaccharides and carrier proteins. “Polysaccharides,” as used herein, include, without limitation, saccharides comprising a plurality of repeating units, including, but not limited to polysaccharides having 50 or more repeat units, and oligosaccharides having 50 or less repeating units. Typically, polysaccharides have from about 50, 55, 60, 65, 70, 75, 80, 85, 90, or 95 repeating units to about 2,000 or more repeating units, and preferably from about 100, 150, 200, 250, 300, 350, 400, 500, 600, 700, 800, 900 or 1000 repeating units to about, 100, 1200, 1300, 1400, 1500, 1600, 1700, 1800, or 1900 repeating units. Oligosaccharides typically have about from about 6, 7, 8, 9, or 10 repeating units to about 15, 20, 25, 30, or 35 to about 40 or 45 repeating units. Suitable carrier proteins typically include bacterial toxins that are immunologically effective carriers that have been rendered safe by chemical or genetic means for administration to a subject. Examples include inactivated bacterial toxins such as diphtheria toxoid, CRM197, tetanus toxoid, pertussis toxoid, E. coli LT, E. coli ST, and exotoxin A from Pseudomonas aeruginosa. Bacterial outer membrane proteins such as, outer membrane complex c (OMPC), porins, transferrin binding proteins, pneumolysis, pneumococcal surface protein A (PspA), pneumococcal adhesion protein (PsaA), or pneumococcal surface proteins BVH-3 and BVH-11 can also be used. Other carrier proteins, such as protective antigen (PA) of Bacillus anthracis and detoxified edema factor (EF) and lethal factor (LF) of Bacillus anthracis, ovalbumin, keyhole limpet hemocyanin (KLH), human serum albumin, bovine serum albumin (BSA) and purified protein derivative of tuberculin (PPD) can also be used.

The quality of lyophilized vaccine components can be assessed and determined by their ability to form a conjugate vaccine. For example, the quality of lyophilized polysaccharides can be determined by their ability to couple or conjugate to a carrier protein. Similarly, the quality of lyophilized carrier proteins can be determined by their ability to couple or conjugate to a polysaccharide. Various methods are known in the art to conjugate a polysaccharide to a carrier protein and the conjugation efficiency can be determined by various analytical methods including, but not limited to, percentage free protein, percentage free polysaccharide, molecular size distribution, saccharide-to-protein ratio (“SPR”) and yield rate. Exemplary methods for determining conjugation efficiency are described in the Examples.

Additional pharmaceutical substances may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, antispasmodics and muscle contractants including channel blockers, miotics and anticholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, antihypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents.

A more complete listing of pharmaceutical substances and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals,” Edited by Susan Budavari et al., CRC Press, 1996, and the United States Pharmacopeia-25/National Formulary-20, published by the United States Pharmacopeial Convention, Inc., Rockville Md., 2001, all of which are incorporated herein by reference.

Lyophilization may be performed in a container, such as a tube, a bag, a bottle, a tray, a vial (e.g., a glass vial), syringe or any other suitable containers or as a bulk in case of spray freeze-drying. As opposed to vial/container freeze-drying when solution is filled into mentioned above containers and freeze-dried under vacuum, in spray freeze-drying solution is spayed into the cold (below −100° C.) column to form frozen pellets which are dried as a bulk. In spray freeze-drying process dry pellets then filled into any type of containers. The containers may be disposable. Controlled freeze and/or thaw may also be performed in a large scale or small scale. Inventive methods in accordance with the present invention can be carried out using various lyophilizers, such as, commercial-scale lyophilizers, pilot-scale lyophilizers, or laboratory-scale lyophilizers.

### C. Lyophilized Lipid Nanoparticles (LNPs)

The present disclosure provides, among other aspects, methods of stabilizing a lipid nanoparticle (LNP), polyplex and lipoplex formulations upon application of stress, before or when the stress is applied. In some embodiments, the stress includes any stress applied to the formulation when producing, purifying, packing, storing, transporting and using the formulation, such as heat, shear, excessive agitation, membrane concentration polarization (change in charge state), dehydration, freezing stress, drying stress, freeze/thaw stress, nebulization stress, etc. For example, the stress can cause one or more undesired property changes to the formulation, such as an increased amount of impurities, of sub-visible particles, or both, an increase in LNP size, a decrease in encapsulation efficiency, in therapeutic efficacy, or both, and a decrease in tolerability (e.g., an increase in immunogenicity)

In some embodiments, the stress applied is from freezing or lyophilizing a LNP formulation. Accordingly, the disclosure also features a method of freezing or lyophilizing a lipid nanoparticle (LNP) formulation, comprising freezing or lyophilizing a first LNP formulation in the presence of a cryoprotectant to obtain a second LNP formulation. For example, the second LNP formulation has substantially no increase in LNP mean size as compared to the first LNP formulation. For example, the second LNP formulation has an increase in LNP mean size of about 20% or less (e.g., about 15%, about 10%, about 5% or less) as compared to the first LNP formulation. For example, the second LNP formulation has substantially no increase in polydispersity index as compared to the first LNP formulation.

For example, the second LNP formulation has an increase in polydispersity index of about 20% or less (e.g., about 15%, about 10%, about 5% or less) as compared to the first LNP formulation.

In one aspect, the present disclosure relates to a method of producing a lipid nanoparticle (LNP) formulation such that the method can influence and/or dictate physical (e.g., LNP stability), chemical (e.g., nucleic acid stability), and/or biological (e.g. efficacy, intracellular delivery, immunogenicity) properties of the LNP formulation.

In some embodiments, the method of the present disclosure mitigates an undesired property change from the produced lipid nanoparticle (LNP) formulation. In some embodiments, the method of the present disclosure mitigates an undesired property change from the produced lipid nanoparticle (LNP) formulation as compared to the LNP formulation produced by a comparable method (e.g., a method without one or more of the steps as disclosed herein).

In some embodiments, the undesired property change caused by a stress upon the LNP formulation or the LNP therein. In some embodiments, the stress is induced during producing, purifying, packing, storing, transporting, and/or using the LNP formulation. In some embodiments, the stress is heat, shear, excessive agitation, membrane concentration polarization (change in charge state), dehydration, freezing stress, drying stress, stress due to crystallization of excipients during freezing, drying or storage, freeze/thaw stress, and/or nebulization stress. In some embodiments, the stress is induced during freezing or lyophilizing a LNP formulation.

In some embodiments, the undesired property change is a reduction of the physical stability of the LNP formulation. In some embodiments, the undesired property change is an increase of the amount of impurities and/or sub-visible particles, or an increase in the average size of the LNP in the LNP formulation.

In some embodiments, the undesired property change is a reduction of the physical stability of the LNP formulation. In some embodiments, the undesired property change is an increase of the amount of impurities and/or sub-visible particles, or an increase in the average size of the LNP in the LNP formulation.

In some embodiments, the method of the present disclosure mitigates a reduction of the physical stability (e.g., an increase in the average size of the LNP) from the produced LNP formulation as compared to the LNP formulation produced by a comparable method as disclosed herein.

In some embodiments, the LNP formulation produced by the method of the present disclosure has an average LNP diameter being about 99% or less, about 98% or less, about 97% or less, about 96% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less as compared to the average LNP diameter of the LNP formulation produced by a comparable method as disclosed herein.

In some embodiments, the undesired property change is a reduction of the chemical stability of the LNP formulation. In some embodiments, the undesired property change is a reduction of the integrity of the nucleic acid (e.g., RNA (e.g., mRNA)) in the LNP formulation.

In some embodiments, the undesired property change is a reduction of the biological property of the LNP formulation. In some embodiments, the undesired property change is a reduction of efficacy, intracellular delivery, and/or immunogenicity of the LNP formulation.

In some embodiments, the LNP formulation produced by the method of the present disclosure has an efficacy, intracellular delivery, and/or immunogenicity being higher than the efficacy, intracellular delivery, and/or immunogenicity of the LNP formulation produced by a comparable method as disclosed herein.

In some embodiments, the LNP formulation produced by the method of the present disclosure has an efficacy, intracellular delivery, and/or immunogenicity being higher than the efficacy, intracellular delivery, and/or immunogenicity of the LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the LNP formulation produced by the method of the present disclosure exhibits a nucleic acid expression (e.g., the mRNA expression) higher than the nucleic acid expression (e.g., the mRNA expression) of the LNP formulation produced by a comparable method.

### D. Methods of Producing Lipid Nanoparticle (LNP) Formulations

In some embodiments, the LNP formulation produced by the method of the present disclosure exhibits a nucleic acid expression (e.g., the mRNA expression) higher than the nucleic acid expression (e.g., the mRNA expression) of the LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some aspects, the present disclosure provides a method of producing a lipid nanoparticle (LNP) formulation, comprising: (i) providing a LNP suspension comprising a lipid nanoparticle (LNP), wherein the LNP comprises a nucleic acid and an ionizable lipid; and (ii) processing the LNP suspension, thereby forming the LNP formulation.

In some aspects, the present disclosure provides a method of producing a lipid nanoparticle (LNP) composition, the method comprising: (i) mixing an aqueous buffer solution and an organic solution, thereby forming a lipid nanoparticle (LNP) formulation comprising a lipid nanoparticle (LNP) encapsulating a nucleic acid; and (ii) processing the lipid nanoparticle (LNP) formulation, thereby forming the lipid nanoparticle composition; wherein the organic solution comprises an organic solvent-soluble nucleic acid and an ionizable lipid in an organic solvent.

Typical lipid nanoparticle (LNP) formation procedures involve the controlled mixing of hydrophobic lipid components dissolved in an organic solvent such as ethanol with an aqueous buffer solution containing the oligonucleotide to be loaded into the resulting particle. Due to the complexity of mixing, and the various ionic interactions necessary to successfully entrap the oligonucleotide in the particle core, there are a large number of variables at play throughout the particle forming process which can impact the quality, stability, and function of the resultant particle.

In some embodiments, the method includes steps to purify, pH adjust, buffer exchange, and/or concentrate LNPs. For example, the method may include: filtering the LNP suspension. In some embodiments, the filtration removes an organic solvent (e.g., an alcohol or ethanol) from the LNP suspension. In some embodiments, the processing comprises a tangential flow filtration (TFF). In some embodiments, upon removal of the organic solvent (e.g. an alcohol or ethanol), the LNP suspension is converted to a solution buffered at a neutral pH, pH 6.5 to 7.8, pH 6.8 to pH 7.5, preferably, pH 7.0 to pH 7.2 (e.g., a phosphate or HEPES buffer). In some embodiments, the resulting LNP suspension is preferably sterilized before storage or use, e.g., by filtration (e.g., through a 0.1-0.5 pm filter).

In some embodiments, the cryoprotectant is added to the LNP suspension prior to the lyophilization. In some embodiments, the cryoprotectant comprises one or more cryoprotective agents, and each of the one or more cryoprotective agents is independently a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-I-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium chloride, sodium formate, sodium malonate, sodium nitrate, sodium sulfate, or any hydrate thereof), or any combination thereof. In some embodiments, the cryoprotectant comprises sucrose.

An exemplary general method for making lipid nanoparticles is as follows. To achieve size reduction and/or to increase the homogeneity of size in the particles, the skilled person may use the method steps set out below, experimenting with different combinations. Additionally, the skilled person could employ sonication, filtration or other sizing techniques which are used in liquid-based formulations, including suspensions.

The process for making a composition of the invention typically comprises providing an aqueous solution, such as citrate buffer, comprising a biologically active agent (e.g., a nucleic acid) in a first reservoir, providing a second reservoir comprising an organic solution, such as an organic alcohol, for example ethanol, of the lipid(s) and then mixing the aqueous solution with the organic lipid solution. The first reservoir is optionally in fluid communication with the second reservoir. The mixing step is optionally followed by an incubation step, a filtration or dialysis step, and a dilution and/or concentration step. The incubation step comprises allowing the solution from the mixing step to stand in a vessel for about 0 to about 100 hours (preferably about 0 to about 24 hours) at about room temperature and optionally protected from light. In one embodiment, a dilution step follows the incubation step. The dilution step may involve dilution with aqueous buffer (e.g. citrate buffer or pure water) e.g., using a pumping apparatus (e.g. a peristaltic pump). The filtration step may include, for example, ultrafiltration or dialysis. Ultrafiltration comprises concentration of the diluted solution followed by diafiltration, e.g., using a suitable pumping system (e.g. pumping apparatus such as a peristaltic pump or equivalent thereof) in conjunction with a suitable ultrafiltration membrane (e.g. GE Hollow fiber cartridges or equivalent). Dialysis comprises solvent (buffer) exchange through a suitable membrane (e.g. 10,000 mwc snakeskin membrane). In one embodiment, the mixing step provides a clear single phase. In one embodiment, after the mixing step, the organic solvent is removed to provide a suspension of particles, wherein the biologically active agent is encapsulated by the lipid(s).

In one embodiment, the method includes: (a) introducing a first stream comprising an anionic macromolecules (e.g., polynucleic acid) in a first solvent into a microchannel; wherein the microchannel has a first region adapted for flowing one or more streams introduced into the microchannel and a second region for mixing the contents of the one or more streams; (b) introducing a second stream comprising transfection reagent composition in a second solvent in the microchannel to provide first and second streams flowing in the device, wherein the transfection reagent composition comprises an ionizable cationic lipid, a neutral lipid, a sterol and a surfactant and wherein the first and second solvents are not the same; (c) flowing the one or more first streams and the one or more second streams from the first region of the microchannel into the second region of the microchannel; and (d) mixing of the contents of the one or more first streams and the one or more second streams flowing in the second region of the microchannel to provide a third stream comprising lipid nanoparticles with encapsulated anionic macromolecules.

The selection of an organic solvent will typically involve consideration of solvent polarity and the ease with which the solvent can be removed at the later stages of particle formation. The organic solvent, which is also used as a solubilizing agent, is preferably in an amount sufficient to provide a clear single-phase mixture of biologically active agents and lipids. The organic solvent may be selected from one or more (e.g. two) of chloroform, dichloromethane, diethylether, cyclohexane, cyclopentane, benzene, toluene, methanol, and other aliphatic alcohols (e.g. C1 to C8) such as ethanol, propanol, isopropanol, butanol, tert-butanol, iso-butanol, pentanol and hexanol. The mixing step can take place by any number of methods, e.g., by mechanical means such as an impinging jet mixer.

The methods used to remove the organic solvent will typically involve diafiltration or dialysis or evaporation at reduced pressures or blowing a stream of inert gas (e.g. nitrogen or argon) across the mixture.

In other embodiments, the method further comprises adding nonlipid polycations which are useful to effect the transformation of cells using the present compositions. Examples of suitable nonlipid polycations include, but are limited to, hexadimethrine bromide (sold under the brandname POLYBRENE®, from Aldrich Chemical Co., Milwaukee, Wis., USA) or other salts of hexadimethrine. Other suitable polycations include, e.g., salts of poly-L-ornithine, poly-L-arginine, poly-L-lysine, poly-D-lysine, polyallylamine and polyethyleneimine. In certain embodiments, the formation of the lipid nanoparticles can be carried out either in a mono-phase system (e.g. a Bligh and Dyer monophase or similar mixture of aqueous and organic solvents) or in a two-phase system with suitable mixing.

The lipid nanoparticle may be formed in a mono- or a bi-phase system. In a mono-phase system, the cationic lipid(s) and biologically active agent are each dissolved in a volume of the mono-phase mixture. Combining the two solutions provides a single mixture in which the complexes form. In a bi-phase system, the cationic lipids bind to the biologically active agent (which is present in the aqueous phase), and “pull” it into the organic phase. In one embodiment, the lipid nanoparticles are prepared by a method which comprises: (a) contacting the biologically active agent with a solution comprising noncationic lipids and a detergent to form a compound-lipid mixture; (b) contacting cationic lipids with the compound-lipid mixture to neutralize a portion of the negative charge of the biologically active agent and form a charge-neutralized mixture of biologically active agent and lipids; and (c) removing the detergent from the charge-neutralized mixture.

In one group of embodiments, the solution of neutral lipids and detergent is an aqueous solution. Contacting the biologically active agent with the solution of neutral lipids and detergent is typically accomplished by mixing together a first solution of the biologically active agent and a second solution of the lipids and detergent. Preferably, the biologically active agent solution is also a detergent solution. The amount of neutral lipid which is used in the present method is typically determined based on the amount of cationic lipid used, and is typically of from about 0.2 to 5 times the amount of cationic lipid, preferably from about 0.5 to about 2 times the amount of cationic lipid used.

The biologically active agent-lipid mixture thus formed is contacted with cationic lipids to neutralize a portion of the negative charge which is associated with the molecule of interest (or other polyanionic materials) present. The amount of cationic lipids used is typically 3-8 fold more than the calculated molar ratio of negative charge (phosphates).

The methods used to remove the detergent typically involve dialysis. When organic solvents are present, removal is typically accomplished by diafiltration or evaporation at reduced pressures or by blowing a stream of inert gas (e.g. nitrogen or argon) across the mixture.

### E. Lipid Nanoparticles

In an exemplary method, LNPs can be formed, for example, by a rapid process which entails micro-mixing the lipid components dissolved in ethanol with an aqueous solution using a confined volume mixing apparatus. The lipid solution contains one or more cationic lipids, one or more noncationic lipids (e.g., DSPC), PEG-DMG, and optionally cholesterol, at specific molar ratios in ethanol. The aqueous solution may include a sodium citrate or sodium acetate buffered salt solution with pH in the range of 2-6, preferably 3.5-5.5. The two solutions are heated to a temperature in the range of 25° C.-45° C., preferably 30° C.-40° C., and then mixed in a confined volume mixer thereby instantly forming the LNP. When a confined volume T-mixer is used, the T-mixer may have an internal diameter (ID) range from 0.25 to 1.0 mm. The alcohol and aqueous solutions are delivered to the inlet of the T-mixer using programmable syringe pumps, and with a total flow rate from 10-600 mL/minute. The alcohol and aqueous solutions may be combined in the confined-volume mixer with a ratio in the range of 1:1 to 1:3 vol:vol. The combination of ethanol volume fraction, reagent solution flow rates and t-mixer tubing ID utilized at this mixing stage has the potential effect of controlling the particle size of the LNPs between 30 and 300 nm. The resulting LNP suspension is twice diluted into higher pH buffers in the range of 6-8 in a sequential, multi-stage in-line mixing process. For example, for the first dilution, the LNP suspension may be mixed with a buffered solution at a higher pH (pH 6-7.5). The resulting LNP suspension is further mixed with a buffered solution at a higher pH, e.g., 6-8. This later buffered solution is at a temperature in the range of 15-40° C., targeting 16-25° C. The mixed LNPs are held from 30 minutes to 2 hours prior to an anion exchange filtration step. After incubation, the LNP suspension may be filtered. The LNPs may be concentrated and diafiltered via an ultrafiltration process where the alcohol is removed and the buffer is exchanged for the final buffer solution such as phosphate buffered saline or a buffer system suitable for cryopreservation (for example containing sucrose, trehalose or combinations thereof). The ultrafiltration process uses a tangential flow filtration format (TFF). This process may use a membrane nominal molecular weight cutoff range from 30-500 KD, targeting 100 KD. The membrane format can be hollow fiber or flat sheet cassette. The TFF processes with the proper molecular weight cutoff retains the LNP in the retentate and the filtrate or permeate contains the alcohol and final buffer wastes. In one embodiment, the TFF process is a multiple step process with an initial concentration to a lipid concentration of 20-30 mg/mL. Following concentration, the LNP suspension is diafiltered against the final buffer (for example, phosphate buffered saline (PBS) with pH 7-8, 10 mM Tris, 140 mM NaCl with pH 7-8, or 10 mM Tris, 70 mM NaCl, 5 wt % sucrose, with pH 7-8) for 5-20 volumes to remove the alcohol and perform buffer exchange. The material is then concentrated an additional 1-3 fold via ultrafiltration. The final steps of the LNP manufacturing process are to sterile filter the concentrated LNP suspension into a suitable container under aseptic conditions. Following filtration, the vialed LNP product is stored under suitable storage conditions (2° C.-8° C., or −20° C. if frozen formulation).

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

A LNP may include one or more components described herein. In some embodiments, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

In some embodiments, for example, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN® 20], polyoxy ethylene sorbitan [TWEEN® 60], polyoxy ethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidyl glycerols, and phosphatidic acids. Phospholipids also include phospho sphingolipid, such as sphingomyelin. In some embodiments, a phospholipid useful or potentially useful in the present invention is an analog or variant of DSPC.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10 s of nm and 100 s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21 st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo % of the LNP, LNP suspension, lyophilized LNP composition, or LNP formulation of the present disclosure is longer than the Txo % of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T1/2 of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T1/2 of the LNP, LNP suspension, lyophilized LNP composition, or LNP formulation of the present disclosure is longer than the T1/2 of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T8o %” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

### F. Lipids

Suitable ionizable lipids for the methods of the present disclosure are further disclosed herein. The lipid component of a LNP may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another particular embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (i.e. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

In some embodiments, the ionizable lipid is a compound of Formula (IL-1):

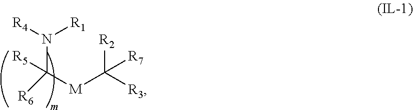

or their N-oxides, or salts or isomers thereof, wherein: Ri is selected from the group consisting of C5-30 alkyl, C5-20 alkenyl, —R*YR″, —YR″, and —R″M′R′; R2 and R3 are independently selected from the group consisting of H, C1-14 alkyl, C2-14 alkenyl, —R*YR″, —YR″, and —R*OR″, or R2 and R3, together with the atom to which they are attached, form a heterocycle or carbocycle; R4 is selected from the group consisting of hydrogen, a C3-6 carbocycle, —(CH2)nQ, —(CH2)nCHQR, —CHQR, —CQ(R)2, and unsubstituted C1-6 alkyl, where Q is selected from a carbocycle, heterocycle, —OR, —O(CH₂)nN(R)2, —C(0)OR, —0C(0)R, —CX3, —CX2H, —CXH2, —CN, —N(R)2, —C(0)N(R)2, —N(R)C(0)R, —N(R)S(0)₂R, —N(R)C(0)N(R)2, —N(R)C(S)N(R)2, —N(R)Re, N(R)S(0)₂R8, —O(CH₂)nOR, —N(R)C(═NR9)N(R)₂, —N(R)C(═CHR9)N(R)₂, —OC(0)N(R)_{2J}—N(R)C(0)OR, —N(OR)C(0)R, —N(0R)S(0)₂R, —N(0R)C(0)OR, —N(0R)C(0)N(R)₂, —N(OR)C(S)N(R)2, —N(OR)C(═NR9)N(R)₂, —N(OR)C(═CHR9)N(R)₂, —C(═NR9)N(R)₂, —C(═NR9)R, —C(0)N(R)0R, and —C(R)N(R)2C(0)0R, and each n is independently selected from 1, 2, 3, 4, and 5; each R5 is independently selected from the group consisting of C1-3 alkyl, C2-3 alkenyl, and H; each Re is independently selected from the group consisting of C1-3 alkyl, C2-3 alkenyl, and H; M and M′ are independently selected from —C(0)0-, —OC(O)—, —0C(0)-M″-C(0)0-, —C(0)N(R′)—, —N(R′)C(0)-, —C(O)—, —C(S)—, —C(S)S—, —SC(S)—, —CH(OH)—, —P(0)(0R′)0-, —S(0)₂—, —S—S—, an aryl group, and a heteroaryl group, in which M″ is a bond, C1-13 alkyl or C2-13 alkenyl; R7 is selected from the group consisting of C1-3 alkyl, C2-3 alkenyl, and H; Re is selected from the group consisting of C3-6 carbocycle and heterocycle; R9 is selected from the group consisting of H, CN, NO2, Ci-6 alkyl, —OR, —S(0)₂R, —S(0)₂N(R)₂, C₂₋₆ alkenyl, C3-6 carbocycle and heterocycle; each R is independently selected from the group consisting of C1-3 alkyl, C₂₋₃ alkenyl, and H; each R′ is independently selected from the group consisting of Ci-is alkyl, C₂-is alkenyl, —R*YR″, —YR″, and H; each R″ is independently selected from the group consisting of C3-15 alkyl and C3-15 alkenyl; each R* is independently selected from the group consisting of Ci-i₂ alkyl and C₂₋ᵢ₂ alkenyl; each Y is independently a C3-6 carbocycle; each X is independently selected from the group consisting of F, Cl, Br, and I; and m is selected from 5, 6, 7, 8, 9, 10, 11, 12, and 13; and wherein when R₄ is —(CH₂)ₙQ, —(CH₂)nCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid. As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerCl4 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid. In some embodiments, the PEG-modified lipids are a modified form of PEG DMG. In some embodiments, the PEG-modified lipid is PEG lipid with the formula (IV):

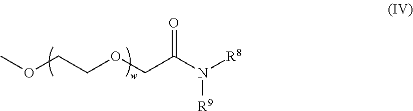

wherein R⁸ and R⁹ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

### G. Polynucleotides and Nucleic Acids

In some embodiments, a LNP includes one or more polynucleotide or nucleic acid (e.g., ribonucleic acid or deoxyribonucleic acid). The term “polynucleotide,” in its broadest sense, includes any compound and/or substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In some embodiments, a therapeutic and/or prophylactic is an RNA. RNAs useful in the compositions and methods described herein can be selected from the group consisting of, but are not limited to, shortmers, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), self-amplifying RNA (saRNA), and mixtures thereof. In certain embodiments, the RNA is an mRNA.

In certain embodiments, a therapeutic and/or prophylactic is an mRNA. An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

In other embodiments, a therapeutic and/or prophylactic is an siRNA. An siRNA may be capable of selectively knocking down or down regulating expression of a gene of interest. For example, an siRNA could be selected to silence a gene associated with a particular disease, disorder, or condition upon administration to a subject in need thereof of a LNP including the siRNA. An siRNA may comprise a sequence that is complementary to an mRNA sequence that encodes a gene or protein of interest. In some embodiments, the siRNA may be an immunomodulatory siRNA.

In some embodiments, a therapeutic and/or prophylactic is an shRNA or a vector or plasmid encoding the same. An shRNA may be produced inside a target cell upon delivery of an appropriate construct to the nucleus. Constructs and mechanisms relating to shRNA are well known in the relevant arts.

Nucleic acids and polynucleotides useful in the disclosure typically include a first region of linked nucleosides encoding a polypeptide of interest (e.g., a coding region), a first flanking region located at the 5′-terminus of the first region (e.g., a 5-UTR), a second flanking region located at the 3′-terminus of the first region (e.g., a 3-UTR), at least one 5′-cap region, and a 3′-stabilizing region. In some embodiments, a nucleic acid or polynucleotide further includes a poly-A region or a Kozak sequence (e.g., in the 5′-UTR). In some cases, polynucleotides may contain one or more intronic nucleotide sequences capable of being excised from the polynucleotide. In some embodiments, a polynucleotide or nucleic acid (e.g., an mRNA) may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-0-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine).

Generally, the shortest length of a polynucleotide can be the length of the polynucleotide sequence that is sufficient to encode for a dipeptide. In another embodiment, the length of the polynucleotide sequence is sufficient to encode for a tripeptide. In another embodiment, the length of the polynucleotide sequence is sufficient to encode for a tetrapeptide. In another embodiment, the length of the polynucleotide sequence is sufficient to encode for a pentapeptide. In another embodiment, the length of the polynucleotide sequence is sufficient to encode for a hexapeptide. In another embodiment, the length of the polynucleotide sequence is sufficient to encode for a heptapeptide. In another embodiment, the length of the polynucleotide sequence is sufficient to encode for an octapeptide. In another embodiment, the length of the polynucleotide sequence is sufficient to encode for a nonapeptide. In another embodiment, the length of the polynucleotide sequence is sufficient to encode for a decapeptide.

In some cases, a polynucleotide is greater than 30 nucleotides in length. In another embodiment, the polynucleotide molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 50 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

In some embodiments, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

Nucleic acids and polynucleotides may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

Nucleic acids and polynucleotides may include one or more alternative components, as described herein, which impart useful properties including increased stability and/or the lack of a substantial induction of the innate immune response of a cell into which the polynucleotide is introduced. For example, an alternative polynucleotide or nucleic acid exhibits reduced degradation in a cell into which the polynucleotide or nucleic acid is introduced, relative to a corresponding unaltered polynucleotide or nucleic acid. These alternative species may enhance the efficiency of protein production, intracellular retention of the polynucleotides, and/or viability of contacted cells, as well as possess reduced immunogenicity.

Polynucleotides and nucleic acids may be naturally or non-naturally occurring. Polynucleotides and nucleic acids may include one or more modified (e.g., altered or alternative) nucleobases, nucleosides, nucleotides, or combinations thereof. The nucleic acids and polynucleotides useful in a LNP can include any useful modification or alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations according to the present disclosure may be alterations of ribonucleic acids (RNAs) to deoxyribonucleic acids (DNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

Polynucleotides and nucleic acids may or may not be uniformly altered along the entire length of the molecule. For example, one or more or all types of nucleotide (e.g., purine or pyrimidine, or any one or more or all of A, G, U, C) may or may not be uniformly altered in a polynucleotide or nucleic acid, or in a given predetermined sequence region thereof. In some instances, all nucleotides X in a polynucleotide (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some instances, nucleic acids do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc., and/or 3) termination or reduction in protein translation.

The nucleic acids can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, vectors). In some embodiments, the nucleic acids may include one or more messenger RNAs (mRNAs) having one or more alternative nucleoside or nucleotides (i.e., alternative mRNA molecules).

The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

Alternative nucleotide base pairing encompasses not only the standard adenine-thymine, adenine-uracil, or guanine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary nonstandard base structures. One example of such non-standard base pairing is the base pairing between the alternative nucleotide inosine and adenine, cytosine, or uracil.

In some embodiments, the nucleobase is an alternative uracil. Exemplary nucleobases and nucleosides having an alternative uracil include pseudouridine (4), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5 s2U), 5-aminomethyl-2-thio-uracil (nmVu), 5-methylaminomethyl-uracil (mnm5U), 5-methylaminomethyl-2-thio-uracil (mnmVu), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnmVu), 5-propynyl-uracil, 1-propynyl-pseudouracil, 5-taurinomethyl-uracil (xm5U), 1-taurinomethyl-pseudouridine, 5-taurinomethyl-2-thio-uracil(xm5 s2U), 1-taurinomethyl-4-thio-pseudouridine, 5-methyl-uracil (m5U, i.e., having the nucleobase deoxythymine), 1-methyl-pseudouridine (mV), 5-methyl-2-thio-uracil (m5 s2U), 1-methyl-4-thio-pseudouridine (m xj/), 4-thio-1-methyl-pseudouridine, 3-methyl-pseudouridine (m \|/), 2-thio-1-methyl-pseudouridine, 1-methyl-1-deaza-pseudouridine, 2-thio-I-methyl-1-deaza-pseudouridine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, NI-methyl-pseudouridine, 3-(3-amino-3-carboxypropyl)uracil (acp U), I-methyl-3-(3-amino-3-carboxypropyl)pseudouridine (acp ψ), 5-(isopentenylaminomethyl)uracil (inm5U), 5-(isopentenylaminomethyl)-2-thio-uracil (inm5 s2U), 5,2′-0-dimethyl-uridine (m5Um), 2-thio-2′-0_methyl-uridine (s2Um), 5-methoxycarbonylmethyl-2′-0-methyl-uridine (mem Um), 5-carbamoylmethyl-2′-0-methyl-uridine (ncm5Um), 5-carboxymethylaminomethyl-2′-0-methyl-uridine (cmnm5Um), 3,2′-0-dimethyl-uridine (m Um), and 5-(isopentenylaminomethyl)-2′-0-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methy 1-pseudoisocytidine, 4-thio-1-methyl-1-deaza-pseudoisocytidine, 1-methyl-1-deaza-pseudoisocytidine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-0-dimethyl-cytidine (m5Cm), N4-acetyl-2′-0-methyl-cytidine (ac4Cm), N4,2′-0-dimethyl-cytidine (m4Cm), 5-formyl-2′-0-methyl-cytidine (f5Cm), N4,N4,2′-0-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (mI A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-0-dimethyl-adenosine (m6Am), N6,N6,2′-0-trimethyl-adenosine (m62Am), 1,2′-0-dimethyl-adenosine (mI Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (1), 1-methyl-inosine (mil), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQI), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2,N2-dimethyl-guanine (m22G), N2,7-dimethyl-guanine (m2,7G), N2,N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-0-methyl-guanosine (m2Gm), N2,N2-dimethyl-2′-0-methyl-guanosine (m22Gm), 1-methyl-2′-0-methyl-guanosine (mIGm), N2,7-dimethyl-2′-0-methyl-guanosine (m2,7Gm), 2′-0-methyl-inosine (Im), 1,2′-0-dimethyl-inosine (mIIm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[I,5-a] I,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

A polynucleotide (e.g., an mRNA) may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-0-methylated. 5′-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (lpswich, MA) may be used with a-thio-guanosine nucleotides according to the manufacturer's instructions to create a phosphorothioate linkage in the 5′-ppp-5′ cap.

Additional alternative guanosine nucleotides may be used such as a-methyl-phosphonate and seleno-phosphate nucleotides. Additional alterations include, but are not limited to, 2′-0-methylation of the ribose sugars of 5-terminal and/or 5′-anteterminal nucleotides of the polynucleotide (as mentioned above) on the 2′-hydroxy group of the sugar. Multiple distinct 5′-cap structures can be used to generate the 5′-cap of a polynucleotide, such as an mRNA molecule.

Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide. For example, the Anti-Reverse Cap Analog (ARCA) cap contains two guanosines linked by a 5′-5′-triphosphate group, wherein one guanosine contains an N7-methyl group as well as a 3′-0-methyl group (i.e., N7,3′-0-dimethyl-guanosine-5′-triphosphate-5′-guanosine, m7G-3′mppp-G, which may equivalently be designated 3′ 0-Me-m7G(5′)ppp(5′)G). The 3′-0 atom of the other, unaltered, guanosine becomes linked to the 5′-terminal nucleotide of the capped polynucleotide (e.g., an mRNA). The N7- and 3′-0-methylated guanosine provides the terminal moiety of the capped polynucleotide (e.g., mRNA). Another exemplary cap is mCAP, which is similar to ARCA but has a 2′-0-methyl group on guanosine (i.e., N7,2′-0-dimethyl-guanosine-5′-triphosphate-5′-guanosine, m7Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G(5)ppp(5′)G and a N7-(4-chlorophenoxyethyl)-m3′-OG(5)ppp(5′)G cap analog (see, e.g., the various cap analogs and the methods of synthesizing cap analogs described in Kore et al. Bioorganic & Medicinal Chemistry 2013 21: 4570-4574; the cap structures of which are herein incorporated by reference). In other instances, a cap analog useful in the polynucleotides of the present disclosure is a 4-chloro/bromophenoxy ethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, recombinant Vaccinia Virus Capping Enzyme and recombinant 2′-0-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-0-methyl. Such a structure is termed the CapI structure. This cap results in a higher translational-competency, cellular stability, and a reduced activation of cellular pro-inflammatory cytokines, as compared, e.g., to other 5′ cap analog structures known in the art. Other exemplary cap structures include 7 mG(5′)ppp(5′)N,pN2p (Cap 0), 7 mG(5′)ppp(5′)NImpNp (Cap 1), 7 mG(5′)-ppp(5′)NImpN2mp (Cap 2), and m(7)Gpppm(3)(6,6,2′)Apm(2′)Apm(2′)Cpm(2)(3,2′)Up (Cap 4).

Because the alternative polynucleotides may be capped post-transcriptionally, and because this process is more efficient, nearly 100% of the alternative polynucleotides may be capped. This is in contrast to −80% when a cap analog is linked to a polynucleotide in the course of an in vitro transcription reaction. 5′-terminal caps may include endogenous caps or cap analogs. A 5′-terminal cap may include a guanosine analog. Useful guanosine analogs include inosine, NI-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5′-cap. A modification on the 5′-cap may increase the stability of polynucleotide, increase the half-life of the polynucleotide, and could increase the polynucleotide translational efficiency. The modified 5′-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

A 5′-UTR may be provided as a flanking region to polynucleotides (e.g., mRNAs). A 5-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

To alter one or more properties of a polynucleotide (e.g., mRNA), 5′-UTRs which are heterologous to the coding region of an alternative polynucleotide (e.g., mRNA) may be engineered. The polynucleotides (e.g., mRNA) may then be administered to cells, tissue or organisms and outcomes such as protein level, localization, and/or half-life may be measured to evaluate the beneficial effects the heterologous 5′-UTR may have on the alternative polynucleotides (mRNA). Variants of the 5′-UTRs may be utilized wherein one or more nucleotides are added or removed to the termini, including A, T, C or G. 5′-UTRs may also be codon-optimized, or altered in any manner described herein.

Polynucleotides (e.g., mRNAs) may include a stem loop such as, but not limited to, a histone stem loop. The stem loop may be a nucleotide sequence that is about 25 or about 26 nucleotides in length. The histone stem loop may be located 3′-relative to the coding region (e.g., at the 3′-terminus of the coding region). As a non-limiting example, the stem loop may be located at the 3′-end of a polynucleotide described herein. In some cases, a polynucleotide (e.g., an mRNA) includes more than one stem loop (e.g., two stem loops). A stem loop may be located in a second terminal region of a polynucleotide. As a non-limiting example, the stem loop may be located within an untranslated region (e.g., 3′-UTR) in a second terminal region. In some cases, a polynucleotide such as, but not limited to mRNA, which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a polynucleotide such as, but not limited to mRNA, which includes the histone stem loop may be stabilized by an alteration to the 3′-region of the polynucleotide that can prevent and/or inhibit the addition of oligio(U). In yet other cases, a polynucleotide such as, but not limited to mRNA, which includes the histone stem loop may be stabilized by the addition of an oligonucleotide that terminates in a 3′-deoxynucleoside, 2′,3′-dideoxynucleoside 3′-0-methylnucleosides, 3-0-ethylnucleosides, 3′-arabinosides, and other alternative nucleosides known in the art and/or described herein. In some instances, the polynucleotides of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5′-cap structure. The 5′-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence.

Polynucleotides may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

A polynucleotide or nucleic acid (e.g., an mRNA) may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an alternative polynucleotide molecule described herein. In other instances, the poly-A region may be 20, 40, 80, 100, 120, 140 or 160 nucleotides in length on an alternative polynucleotide molecule described herein. In some cases, the poly-A region is designed relative to the length of the overall alternative polynucleotide. This design may be based on the length of the coding region of the alternative polynucleotide, the length of a particular feature or region of the alternative polynucleotide (such as mRNA) or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region.

In certain cases, engineered binding sites and/or the conjugation of polynucleotides (e.g., mRNA) for poly-A binding protein may be used to enhance expression. The engineered binding sites may be sensor sequences which can operate as binding sites for ligands of the local microenvironment of the polynucleotides (e.g., mRNA). As a non-limiting example, the polynucleotides (e.g., mRNA) may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct polynucleotides (e.g., mRNA) may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3′-5′-exonuclease digestion. In some instances, a polynucleotide (e.g., mRNA) may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant polynucleotides (e.g., mRNA) may be assayed for stability, protein production and other parameters including half-life at various time points. It has been discovered that the polyA-G quartet results in protein production equivalent to at least 75% of that seen using a poly-A region of 120 nucleotides alone. In some cases, a polynucleotide (e.g., mRNA) may include a poly-A region and may be stabilized by the addition of a 3′-stabilizing region. The polynucleotides (e.g., mRNA) with a poly-A region may further include a 5′-cap structure. In other cases, a polynucleotide (e.g., mRNA) may include a poly-A-G Quartet. The polynucleotides (e.g., mRNA) with a poly-A-G Quartet may further include a 5′-cap structure. In some cases, the 3′-stabilizing region which may be used to stabilize a polynucleotide (e.g., mRNA) including a poly-A region or poly-A-G Quartet. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxy thymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside. In other cases, a polynucleotide such as, but not limited to mRNA, which includes a polyA region or a poly-A-G Quartet may be stabilized by an alteration to the 3′-region of the polynucleotide that can prevent and/or inhibit the addition of oligio(U). In yet other instances, a polynucleotide such as, but not limited to mRNA, which includes a poly-A region or a poly-A-G Quartet may be stabilized by the addition of an oligonucleotide that terminates in a 3′-deoxynucleoside, 2′,3′-dideoxynucleoside 3-0-methylnucleosides, 3′-0-ethylnucleosides, 3′-arabinosides, and other alternative nucleosides known in the art and/or described herein.

H. Exemplary RNA Sequences FgenI and Chi18-4 model RNA were chosen and designed based on Leija-Martinex et al., with 5′ and 3′ primer annealing sites at the two ends (N. Leija-Martinez et al., The separation between the 5′-3′ ends in long RNA molecules is short and nearly constant. Nucleic Acids Res 42, 13963-13968 (2014)).

The FgenI RNA sequences were derived from a fungal organism named Trichoderma atroviride and are available on the fungal genomics resource database with protein ID 258498.

>RNA sequence derived from HCV IRES
GCCAGCCCCCGAUUGGGGGCGACACUCCACCAUAGAUCACUCCCCUGUGAGGAACUA
CUGUCUUCACGCAGAAAGCGUCUAGCCAUGGCGUUAGUAUGAGUGUCGUGCAGCCUC
CAGGACCCCCCCUCCCGGGAGAGCCAUAGUGGUCUGCGGAACCGGUGAGUACACCGG
AAUUGCCAGGACGACCGGGUCCUUUCUUGGAUCAACCCGCUCAAUGCCUGGAGAUUU
GGGCGUGCCCCCGCGAGACUGCUAGCCGaAGUAGUGUUGGGUCGCGAAAGGCCUUG
UGGUACUGCCUGAUAGGGUGCUUGCGAGUGCCCCGGGAGGUCUCGUAGACCGUGCA
CCAUGAGCACGAAUCCUAAACCUCAAAGAAAAACCAAACGUAACACCAACCGCCGCCCA
CAGGACGUCUGAGGCGGCCGCCGCCUCAGACGUCcuGUGGGUUUAUUGCAUCCCGC
(SEQ ID NO: 1)
>DNA transcript, derived from HCV IRES (T7 promoter in bold; site in italics)
5'-
GGATCCTAATACGACTCACTATAGCCAGCCCCCGATTGGGGGCGACACTCCACCATAG
ATCACTCCCCTGTGAGGAACTACTGTCTTCACGCAGAAAGCGTCTAGCCATGGCGTTAG
TATGAGTGTCGTGCAGCCTCCAGGACCCCCCCTCCCGGGAGAGCCATAGTGGTCTGCG
GAACCGGTGAGTACACCGGAATTGCCAGGACGACCGGGTCCTTTCTTGGATCAACCCGC
TCAATGCCTGGAGATTTGGGCGTGCCCCCGCGAGACTGCTAGCCGAGTAGTGTTGGGT
CGCGAAAGGCCTTGTGGTACTGCCTGATAGGGTGCTTGCGAGTGCCCCGGGAGGTCTC
GTAGACCGTGCACCATGAGCACGAATCCTAAACCTCAAAGAAAAACCAAACGTAACACCA
ACCGCCGCCCACAGGACGTCTGAGGCGGCCGCCGCCTCAGACGTCCTGTGGGTTTATT
GCATCCCGCCTCGAGTCTAGA-3' (SEQ ID NO: 2)
>FgenI model RNA synthesized RNA sequence
UCUCUCGCUAUCUCGGAAUCGAGGGGUCUGGCCUACGCUGCGAUUGCUUCUUCAGAG
CAUCCUUCUUCAGCCUUGUGCCCUCUACAGUGGCAGCCUCACACAAACUAUCAACAUG
GCUUCCGAAUCCAGGCUCUACCAGAUCUCCGGCGAGACCAAGUCUCAUCUGCUCAAG
UUCCGCUUGACGACAUCUCGGGCCAGCAAACCUCAGGCAGUGAUUUAUUUGAUUGAU
AAAAACACUCACGAAAUCCGCCAAGACGAUGACAAGACUGUAUACACCUCCCUCGACG
AGAUAGCCGACGACCUCCCCGACAGCACCCCUCGAUUCAUCUUCCUCAGCUAUCCCU
UGACGAUGCCCGAUGGACGGCUAUCCGUGCCUUACACCAUGAUCUACUACCUCCCCA
UCAACUGCAAUGCCGCAACAAGGAUGCUCUACGCCGGCGCAAAAGAGCUGAUACGCAA
CACGGCCGAGGUGAACAAGGUUAUCGAUAUAGAGUCUGCAGAGGAUCUGGAAGAUAU
UCCAAAGCAGUUGAGCGGAUAGAAAUAAUAAUAAAAGAAAUAACAUGUUCGCUAUGCC
(SEQ ID NO: 3)
>FgenI model RNA synthesized RNA sequence, with primer annealing sites
UCCACCAUGAAUCACUCCUCUCUCGCUAUCUCGGAAUCGAGGGGUCUGGCCUACGCU
GCGAUUGCUUCUUCAGAGCAUCCUUCUUCAGCCUUGUGCCCUCUACAGUGGCAGCCU
CACACAAACUAUCAACAUGGCUUCCGAAUCCAGGCUCUACCAGAUCUCCGGCGAGACC
AAGUCUCAUCUGCUCAAGUUCCGCUUGACGACAUCUCGGGCCAGCAAACCUCAGGCA
GUGAUUUAUUUGAUUGAUAAAAACACUCACGAAAUCCGCCAAGACGAUGACAAGACUG
UAUACACCUCCCUCGACGAGAUAGCCGACGACCUCCCCGACAGCACCCCUCGAUUCAU
CUUCCUCAGCUAUCCCUUGACGAUGCCCGAUGGACGGCUAUCCGUGCCUUACACCAU
GAUCUACUACCUCCCCAUCAACUGCAAUGCCGCAACAAGGAUGCUCUACGCCGGCGC
AAAAGAGCUGAUACGCAACACGGCCGAGGUGAACAAGGUUAUCGAUAUAGAGUCUGCA
GAGGAUCUGGAAGAUAUUCCAAAGCAGUUGAGCGGAUAGAAAUAAUAAUAAAAGAAAU
AACAUGUUCGCUAUGCCGAAAAACCAAACGUAACACC (SEQ ID NO: 4)
>DNA template of FgenI model RNA, with primer annealing sites
TCCACCATGAATCACTCCTCTCTCGCTATCTCGGAATCGAGGGGTCTGGCCTACGCTGC
GATTGCTTCTTCAGAGCATCCTTCTTCAGCCTTGTGCCCTCTACAGTGGCAGCCTCACAC
AAACTATCAACATGGCTTCCGAATCCAGGCTCTACCAGATCTCCGGCGAGACCAAGTCT
CATCTGCTCAAGTTCCGCTTGACGACATCTCGGGCCAGCAAACCTCAGGCAGTGATTTAT
TTGATTGATAAAAACACTCACGAAATCCGCCAAGACGATGACAAGACTGTATACACCTCC
CTCGACGAGATAGCCGACGACCTCCCCGACAGCACCCCTCGATTCATCTTCCTCAGCTA
TCCCTTGACGATGCCCGATGGACGGCTATCCGTGCCTTACACCATGATCTACTACCTCC
CCATCAACTGCAATGCCGCAACAAGGATGCTCTACGCCGGCGCAAAAGAGCTGATACGC
AACACGGCCGAGGTGAACAAGGTTATCGATATAGAGTCTGCAGAGGATCTGGAAGATAT
TCCAAAGCAGTTGAGCGGATAGAAATAATAATAAAAGAAATAACATGTTCGCTATGCCGA
AAAACCAAACGTAACACC (SEQ ID NO: 5)
>Chi18-4 model RNA synthesized RNA sequence
UACGCCAGACAAUAGAUUCAGCUCGAAUAAUGAAGCCGAUUGGUCACUGAUUAACUUG
GUGUAUCUACUGGGUAGCAAAGGACCGAUCUUCACCUCGCAUGACCGUUGAAUGUGG
CGCAAUUCUAGUACUUGAAUGGCCUGGCCAAGCUUAUAGAACAAUCGAGACGAAUUGA
ACACCACUAUCAAAAUCCAAAUUAUGCGGUUUUCAAUCAUACAUGUGGCUUUAUGGCU
GGCCAUGGCGGCCGGUUCAUUUACAGCUAACGCUUACGGAGCUGUUCGAUGCGUCAU
GUAUCUCACAGGGCAACACGUGGUAGUCCCUUCAGAACCUCAUCUCGUGGAUUCCAU
AACCCAUUUGAUACUGGCUUUUAUGCGCUCUGAUGUCUUCAAUGUGGACAAAACGCC
UGCUGAAUUCCCGCUCUUCGCAUCAGUUGCUGAGACGCGCGAGAAGUUCAACGCAAA
UACCAAGAUCAUGGUCGCAAUCGGCGGUUGGGGAGACGCAGGAUUUGAAGAAGCUGC
GCGUGACGAUUCAUCGAGAAAGCGGUGGGCUGGCCACGUCAAGGCCAUGGUUGACCA
GACAGGAGCCGAUGGCAUUGACAUUGACUGGGAAUAUCCGGGAGGGAACCGUGACGA
UUAUAAACUCAUUCCGAACUCUCAGCGGGAAUGGGAGAUUCAGGCAUUCGUGCUCCU
UCUUCAAGAAUUGCGUUCCGCCUUGGGACAAACGAAACUGCUCUCAAUUGCGGUGCC
AGCGCUGGAACGAGAUUUGAUGGCCUUCACAAACUCGACUAUUCCGUCCAUUGUGGA
UCAAGUUGACUUUAUCAGUGUAAUGACUUACGACAUGAUGAACCGACGCGACAACGUU
GUCAAGCACCACAGCGGCGUGGCUGACUCCUGGGAAGCAAUGAAGCGAUAUAUAGAU
CGCGGCGUCCCUCCGCACAAGCUGAACUUUGGACUUGGUUACUAUGCCAAAUGGUUC
AUGACUGAACAAUGCGAUGUACAGCACCCAUUAGGAUGCCGCACUCAACUGCUAGAAG
ACCCCGCCAAUGGAGCCGAUCUUGGCAAGACUGCAGCUUUUAGCUGGCACGACGAGG
UUCCCGCAGAAAUGGCCAAUUCCUUUGGCAAAGCCCAUGCUCAUGGCCGCUACUAUG
AAGAUGGAAGCUACGGCUAUUGGGACGAUGAAGAGAAGAGAUGGUGGUCCUACGACA
CGCCUCUCACCAUCAAAGCCAAAGUGCCUCGGCUUCUCGGCGAGCUGCAGCUGGGCG
GGGUGUUUGCCUGGGGGCUGGGCGAGGACGCUCCGCAGUUUCUGCACCUGAAGGCG
ACUGUUGACGGCAUUCGGGUUCUGCGCGGAGAUGACGAUGCUGUGAAGGAUGAGCU
GUAACAAGCAGCGGCGUGUGUACGUCUGGAGUUUGGCGACAUCAUUGAAGGCGUGG
GAUGCAAUUCAAGCCAUUGAUACUGCAGAUAAGUUGAAGCAGCAACAGCCAAAACUCU
GCACGUGGCCCGAGACUUCCGUCUUAUAGCAGGGAACAGACAGGAUGGUGCUGACGG
AAUAUGCAGAGCUCGAGCUCAACCGGUAGGCUAGCUUGAAAACCCCAUGAGCUAGCA
GUUAACCUUGCAGUUAAGGGUAACCAGAGCUUAAACACGUCUGAUUUGGUGCAAUGA
AUACAGGCUGC (SEQ ID NO: 6)
>Chi18-4 model RNA synthesized RNA sequence, primer annealing sites in bold
UCCACCAUGAAUCACUCCUACGCCAGACAAUAGAUUCAGCUCGAAUAAUGAAGCCGAU
UGGUCACUGAUUAACUUGGUGUAUCUACUGGGUAGCAAAGGACCGAUCUUCACCUCG
CAUGACCGUUGAAUGUGGCGCAAUUCUAGUACUUGAAUGGCCUGGCCAAGCUUAUAG
AACAAUCGAGACGAAUUGAACACCACUAUCAAAAUCCAAAUUAUGCGGUUUUCAAUCA
UACAUGUGGCUUUAUGGCUGGCCAUGGCGGCCGGUUCAUUUACAGCUAACGCUUACG
GAGCUGUUCGAUGCGUCAUGUAUCUCACAGGGCAACACGUGGUAGUCCCUUCAGAAC
CUCAUCUCGUGGAUUCCAUAACCCAUUUGAUACUGGCUUUUAUGCGCUCUGAUGUCU
UCAAUGUGGACAAAACGCCUGCUGAAUUCCCGCUCUUCGCAUCAGUUGCUGAGACGC
GCGAGAAGUUCAACGCAAAUACCAAGAUCAUGGUCGCAAUCGGCGGUUGGGGAGACG
CAGGAUUUGAAGAAGCUGCGCGUGACGAUUCAUCGAGAAAGCGGUGGGCUGGCCACG
UCAAGGCCAUGGUUGACCAGACAGGAGCCGAUGGCAUUGACAUUGACUGGGAAUAUC
CGGGAGGGAACCGUGACGAUUAUAAACUCAUUCCGAACUCUCAGCGGGAAUGGGAGA
UUCAGGCAUUCGUGCUCCUUCUUCAAGAAUUGCGUUCCGCCUUGGGACAAACGAAAC
UGCUCUCAAUUGCGGUGCCAGCGCUGGAACGAGAUUUGAUGGCCUUCACAAACUCGA
CUAUUCCGUCCAUUGUGGAUCAAGUUGACUUUAUCAGUGUAAUGACUUACGACAUGA
UGAACCGACGCGACAACGUUGUCAAGCACCACAGCGGCGUGGCUGACUCCUGGGAAG
CAAUGAAGCGAUAUAUAGAUCGCGGCGUCCCUCCGCACAAGCUGAACUUUGGACUUG
GUUACUAUGCCAAAUGGUUCAUGACUGAACAAUGCGAUGUACAGCACCCAUUAGGAUG
CCGCACUCAACUGCUAGAAGACCCCGCCAAUGGAGCCGAUCUUGGCAAGACUGCAGC
UUUUAGCUGGCACGACGAGGUUCCCGCAGAAAUGGCCAAUUCCUUUGGCAAAGCCCA
UGCUCAUGGCCGCUACUAUGAAGAUGGAAGCUACGGCUAUUGGGACGAUGAAGAGAA
GAGAUGGUGGUCCUACGACACGCCUCUCACCAUCAAAGCCAAAGUGCCUCGGCUUCU
CGGCGAGCUGCAGCUGGGGGGGGUGUUUGCCUGGGGGCUGGGCGAGGACGCUCCG
CAGUUUCUGCACCUGAAGGCGACUGUUGACGGCAUUCGGGUUCUGCGCGGAGAUGA
CGAUGCUGUGAAGGAUGAGCUGUAACAAGCAGCGGCGUGUGUACGUCUGGAGUUUG
GCGACAUCAUUGAAGGCGUGGGAUGCAAUUCAAGCCAUUGAUACUGCAGAUAAGUUG
AAGCAGCAACAGCCAAAACUCUGCACGUGGCCCGAGACUUCCGUCUUAUAGCAGGGA
ACAGACAGGAUGGUGCUGACGGAAUAUGCAGAGCUCGAGCUCAACCGGUAGGCUAGC
UUGAAAACCCCAUGAGCUAGCAGUUAACCUUGCAGUUAAGGGUAACCAGAGCUUAAAC
ACGUCUGAUUUGGUGCAAUGAAUACAGGCUGCGAAAAACCAAACGUAACACC (SEQ
ID NO: 7)
>DNA transcript for Chi18-4 model RNA synthesized RNA sequence, primer
annealing sites in bold
TCCACCATGAATCACTCCTACGCCAGACAATAGATTCAGCTCGAATAATGAAGCCGATTG
GTCACTGATTAACTTGGTGTATCTACTGGGTAGCAAAGGACCGATCTTCACCTCGCATGA
CCGTTGAATGTGGCGCAATTCTAGTACTTGAATGGCCTGGCCAAGCTTATAGAACAATCG
AGACGAATTGAACACCACTATCAAAATCCAAATTATGCGGTTTTCAATCATACATGTGGCT
TTATGGCTGGCCATGGCGGCCGGTTCATTTACAGCTAACGCTTACGGAGCTGTTCGATG
CGTCATGTATCTCACAGGGCAACACGTGGTAGTCCCTTCAGAACCTCATCTCGTGGATTC
CATAACCCATTTGATACTGGCTTTTATGCGCTCTGATGTCTTCAATGTGGACAAAACGCCT
GCTGAATTCCCGCTCTTCGCATCAGTTGCTGAGACGCGCGAGAAGTTCAACGCAAATAC
CAAGATCATGGTCGCAATCGGCGGTTGGGGAGACGCAGGATTTGAAGAAGCTGCGCGT
GACGATTCATCGAGAAAGCGGTGGGCTGGCCACGTCAAGGCCATGGTTGACCAGACAG
GAGCCGATGGCATTGACATTGACTGGGAATATCCGGGAGGGAACCGTGACGATTATAAA
CTCATTCCGAACTCTCAGCGGGAATGGGAGATTCAGGCATTCGTGCTCCTTCTTCAAGAA
TTGCGTTCCGCCTTGGGACAAACGAAACTGCTCTCAATTGCGGTGCCAGCGCTGGAACG
AGATTTGATGGCCTTCACAAACTCGACTATTCCGTCCATTGTGGATCAAGTTGACTTTATC
AGTGTAATGACTTACGACATGATGAACCGACGCGACAACGTTGTCAAGCACCACAGCGG
CGTGGCTGACTCCTGGGAAGCAATGAAGCGATATATAGATCGCGGCGTCCCTCCGCACA
AGCTGAACTTTGGACTTGGTTACTATGCCAAATGGTTCATGACTGAACAATGCGATGTAC
AGCACCCATTAGGATGCCGCACTCAACTGCTAGAAGACCCCGCCAATGGAGCCGATCTT
GGCAAGACTGCAGCTTTTAGCTGGCACGACGAGGTTCCCGCAGAAATGGCCAATTCCTT
TGGCAAAGCCCATGCTCATGGCCGCTACTATGAAGATGGAAGCTACGGCTATTGGGACG
ATGAAGAGAAGAGATGGTGGTCCTACGACACGCCTCTCACCATCAAAGCCAAAGTGCCT
CGGCTTCTCGGCGAGCTGCAGCTGGGCGGGGTGTTTGCCTGGGGGCTGGGCGAGGAC
GCTCCGCAGTTTCTGCACCTGAAGGCGACTGTTGACGGCATTCGGGTTCTGCGCGGAG
ATGACGATGCTGTGAAGGATGAGCTGTAACAAGCAGCGGCGTGTGTACGTCTGGAGTTT
GGCGACATCATTGAAGGCGTGGGATGCAATTCAAGCCATTGATACTGCAGATAAGTTGA
AGCAGCAACAGCCAAAACTCTGCACGTGGCCCGAGACTTCCGTCTTATAGCAGGGAACA
GACAGGATGGTGCTGACGGAATATGCAGAGCTCGAGCTCAACCGGTAGGCTAGCTTGAA
AACCCCATGAGCTAGCAGTTAACCTTGCAGTTAAGGGTAACCAGAGCTTAAACACGTCTG
ATTTGGTGCAATGAATACAGGCTGCGAAAAACCAAACGTAACACC (SEQ ID NO: 8)
>RNA FP Stabilization Assay
GGAAUCUCUCUCACGAACUGACGUAAUCUU (SEQ ID NO: 9)

### I. Product Attributes of Frozen or Lyophilized Products

The frozen or lyophilized composition of this invention comprises quality attributes which include, but are not limited to, LNP size, polydispersity index, particle morphology, payload encapsulation, and payload integrity.

“LNP size” shall mean average hydrodynamic diameter of the particle population (e.g. dynamic light scattering to measure z-average).

“Polydispersity index” shall mean measurement of the heterogeneity of a particle population based on size.

“Payload encapsulation” shall mean the fraction of the payload (i.e. a pharmaceutical substance with or without one or more stabilizing agent) associated with the nanoparticles determined using an appropriate analytical method (e.g., spectrophotometry to measure free and total payload content).

“Payload integrity” shall mean fraction of intact payload determined using an appropriate analytical method (e.g., capillary electrophoresis to determine RNA integrity).

This invention provides a method for producing a frozen or lyophilized formulation, wherein the attributes of the frozen or lyophilized formulation are comparable to a control formulation.

In one embodiment, the attributes of the frozen or lyophilized product are maintained over a period of from about 3 months to about 24 months.

It should be understood that the above-described embodiments and the following examples are given by way of illustration, not limitation. Lyophilization methods in accordance with the present invention can be applied to any molecules (e.g., proteins, lipids, nucleic acids, etc.) in general. For example, the molecules used in the following examples can be any proteins, antibodies, nucleic acids, chemical compounds, vaccines, enzymes, polysaccharides, natural products, small molecules, or any other types of molecules. Various changes and modifications within the scope of the present invention will become apparent to those skilled in the art from the present description.

### EXAMPLES

In order that this invention may be better understood, the following examples are set forth. These examples are for purposes of illustration only and are not to be construed as limiting the scope of the invention in any manner. The following Examples illustrate some embodiments of the invention.

### Example 1

In this example concentration of active ingredient (mRNA) was varied in a range of 0.1 to 0.5 mg/mL while stabilizer (sucrose) concentration was in a range between 10 to 400 mg/mL.

TABLE 1
Formulation components

| | Fill | mRNA | Sucrose | Ratio |
|---|---|---|---|---|
| | volume, | concentration, | concentration, | sucrose |
| Formulation# | mL | mg/mL | mg/mL | to mRNA |
| F1 | 0.45 | 0.5 | 10 | 20 |
| F2 | 0.45 | 0.5 | 20 | 40 |
| F3 | 0.45 | 0.5 | 50 | 100 |
| F4 | 0.45 | 0.5 | 103 | 206 |
| F5 | 0.45 | 0.5 | 150 | 300 |
| F6 | 0.45 | 0.5 | 200 | 400 |
| F7 | 0.45 | 0.5 | 300 | 600 |
| F8 | 0.45 | 0.5 | 400 | 800 |
| F9 | 0.3 | 0.2 | 10 | 50 |
| F10 | 0.3 | 0.2 | 20 | 100 |
| F11 | 0.3 | 0.2 | 50 | 250 |
| F12 | 0.3 | 0.2 | 103 | 515 |
| F13 | 0.3 | 0.2 | 150 | 750 |
| F14 | 0.3 | 0.2 | 200 | 1000 |
| F15 | 0.3 | 0.2 | 300 | 1500 |
| F16 | 0.3 | 0.2 | 400 | 2000 |
| F17 | 0.55 | 0.1 | 10 | 100 |
| F18 | 0.55 | 0.1 | 20 | 200 |
| F19 | 0.55 | 0.1 | 50 | 500 |
| F20 | 0.55 | 0.1 | 103 | 1030 |
| F21 | 0.55 | 0.1 | 150 | 1500 |
| F22 | 0.55 | 0.1 | 200 | 2000 |
| F23 | 0.55 | 0.1 | 300 | 3000 |
| F24 | 0.55 | 0.1 | 400 | 4000 |

Note that all formulations contain 7.5 mM or 10 mM Tris as a buffer. Formulation pH is 7.4

Two lyophilization cycles were used to screen these formulations: conservative (Table 2) and improved (Table 3). 2-mL vials were used in both exemplary cycles.

TABLE 2
Conservative lyophilization cycle for
formulations F1-F6, F9-F14, F17-F22

Process parameters

| Process | Conservative | Cumulative |
|---|---|---|
| steps | cycle | Time, hours |
| Freezing | Ramp to −45° C. in | 2.17 |
| including | 130 min | |
| Annealing | Hold at −45° C. for | 3.17 |
| at −10° | 60 min | |
| C. for | Ramp to −10° C. in | 4.33 |
| 180 min | 70 min | |
| | Hold at −10° C. for | 7.33 |
| | 180 min | |
| | Ramp to −45° C. in | 8.50 |
| | 70 min | |
| | Hold at −45° C. for | 9.50 |
| | 60 min | |
| Chill | Temperature_{condenser} | 10.50 |
| condenser | <−60° C.; | |
| and pull | Pressure_{chamber} = | |
| vacuum | 50 mTorr | |
| Primary | Ramp to −29° C. in | 11.03 |
| drying | 32 min | |
| | Hold at −29° C. for | 41.03 |
| | 1800 min | |
| Secondary | Ramp to 30° C. in | 45.95 |
| drying | 295 min | |
| | Hold at 30° C. for | 53.95 |
| | 480 min | |

TABLE 3
Improved lyophilization cycle (with an
annealing step) for formulations F9-F16

Process parameters

| Process | Improved | Cumulative |
|---|---|---|
| steps | cycle | Time, hours |
| Freezing | Ramp to −45° C. in | 2.17 |
| including | 130 min | |
| Annealing | Hold at −45° C. for | 3.17 |
| at −10° | 60 min | |
| C. for | Ramp to −10° C. in | 4.33 |
| 180 min | 70 min | |
| | Hold at −10° C. for | 7.33 |
| | 180 min | |
| | Ramp to −45° C. in | 8.50 |
| | 70 min | |
| | Hold at −45° C. for | 9.50 |
| | 60 min | |
| Chill | Temperature_{condenser} | 10.50 |
| condenser | <−60° C.; | |
| and pull | Pressure_{chamber} = | |
| vacuum | 50 mTorr | |
| Primary | Ramp to −25° C. in | 11.17 |
| drying | 40 min | |
| | Hold at −25° C. for | 21.17 |
| | 600 min | |
| Secondary | Ramp to 30° C. in | 25.75 |
| drying | 275 min | |
| | Hold at 30° C. for | 33.75 |
| | 480 min | |

TABLE 4
Improved lyophilization cycle (without an
annealing step) for formulations F9-F16

Process parameters

| Process | Improved | Cumulative |
|---|---|---|
| steps | cycle | Time, hours |
| Freezing | Ramp to −45° C. in | 2.17 |
| step | 130 min | |
| without | Hold at −45° C. for | 3.17 |
| annealing | 60 min | |
| Chill | Temperature_{condenser} | 4.17 |
| condenser | <−60° C.; | |
| and pull | Pressure_{chamber} = | |
| vacuum | 50 mTorr | |
| Primary | Ramp to −27° C. in | 4.77 |
| drying | 36 min | |
| | Hold at −27° C. for | 11.17 |
| | 420 min | |
| | Hold at −28° C. for | 19.17 |
| | 480 min | |
| Secondary | Ramp to 30° C. in | 24.60 |
| drying | 290 min | |
| | Hold at 30° C. for | 32.60 |
| | 480 min | |

TABLE 5
Improved Lyophilization cycle for formulations F9-F16.

| | Time, | Time, | Cumulative | Vacuum, |
|---|---|---|---|---|
| Cycle design | min | hrs | time, hrs | mTorr |
| Ramp from 20° C. to −45° C. | 130.00 | 2.17 | 2.17 | |
| at 0.5° C./min | | | | |
| Hold at −45° C. for 1 hours | 60.00 | 1.00 | 3.17 | |
| Puul vacuum to 40 mT | 60.00 | 1.00 | 4.17 | 40 |
| Ramp to −27° C. at 0.5° C./min | 26.00 | 0.43 | 4.60 | 40 |
| Hold at −27° C. for 7 hours | 420.00 | 7.00 | 11.60 | 40 |
| Hold at −28° C. for 8 hours | 480 | 8.00 | 19.60 | 40 |
| Ramp to 30° C. at 0.2° C./min | 290.00 | 4.83 | 24.43 | 40 |
| Hold at 30° C. for 8 hours | 480.00 | 8.00 | 32.43 | 40 |

### Example 2

In this example the concentration of the active ingredient (mRNA) was constant and equal to 0.18 mg/mL at a fill volume of 0.3 mL while shelf temperatures and pressures during lyophilization varied.

Three lyophilization cycles were used to confirm robustness of the process and to define a design space for the process parameters described in this Example. 2-mL vials were used in these exemplary cycles.

TABLE 6
Process parameters for target, low limit and high limit cycles for formulations F9-F16

| | Process parameters | | | | | |
|---|---|---|---|---|---|---|
| | Target cycle | | Low limit cycle | | High limit cycle | |
| | Cycle | Cumulative | Cycle | Cumulative | Cycle | Cumulative |
| | decription | Time, | decription | Time, | decription | Time, |
| Process | Loading | hours | Loading | hours | Loading | hours |
| steps | temperature | 5° C. | temperature | 2º C. | temperature | 8° C. |
| Freezing | Ramp to −45º C. | 1.67 | Ramp to −48º C. | 1.67 | Ramp to −42º C. | 1.67 |
| including | in | | in | | in | |
| Annealing | 100 min | | 100 min | | 100 min | |
| | Hold at −45° C. | 2.67 | Hold at −48º C. | 2.67 | Hold at −42° C. | 2.67 |
| | for | | for | | for | |
| | 60 min | | 60 min | | 60 min | |
| | Ramp to −10° C. | 3.83 | Ramp to −13º C. | 3.83 | Ramp to −7° C. in | 3.83 |
| | in | | in | | 70 min | |
| | 70 min | | 70 min | | | |
| | Hold at −10º C. | 6.83 | Hold at −13º C. | 6.83 | Hold at −7° C. for | 6.83 |
| | for | | for | | 180 min | |
| | 180 min | | 180 min | | | |
| | Ramp to −45° C. | 8.00 | Ramp to −48º C. | 8.00 | Ramp to −42º C. | 8.00 |
| | in | | in | | in | |
| | 70 min | | 70 min | | 70 min | |
| | Hold at −45° C. | 9.00 | Hold at −48° C. | 9.00 | Hold at −42º C. | 9.00 |
| | for | | for | | for | |
| | 60 min | | 60 min | | 60 min | |
| Chill | Temperature, | 10.00 | Temperature | 10.00 | Temperature | 10.00 |
| condenser | _{condenser} | | _{condenser} | | _{condenser} | |
| and pull | <−60º C.; | | <−60° C.; | | <−60º C.; | |
| vacuum | Pressure | | Pressure | | Pressure | |
| | _{chamber} | | _{chamber} | | _{chamber} | |
| | = | | = | | = | |
| | 50 mTorr | | 35 mTorr | | 88 mTorr | |
| Primary | Ramp to −25° C. | 10.67 | Ramp to −28° C. | 10.67 | Ramp to −22º C. | 10.67 |
| drying | in | | in | | in | |
| | 40 min | | 40 min | | 40 min | |
| | Hold at −25° C. | 22.67 | Hold at −28º C. | 22.67 | Hold at −22º C. | 22.67 |
| | for 720 min | | for 720 min | | for 720 min | |
| Secondary | Ramp to 35º C. in | 27.67 | Ramp to 32º C. | 27.67 | Ramp to 38° C. in | 27.67 |
| drying | 300 min | | in | | 300 min | |
| | | | 300 min | | | |
| | Hold at 35º C. for | 33.67 | Hold at 32ºC | 33.67 | Hold at 38º C. for | 33.67 |
| | 360 min | | for | | 360 min | |

3600 min

To explore the edge of failure (i.e. when product temperature exceeds the collapse temperature) two high temperature cycles were also performed with formulations F9-F16 (shown in Table 1). 2-mL vials filled with 0.3 mL solution were used in these exemplary cycles (Table 7).

TABLE 7
Process parameters for high temperature cycles for formulations F9-F16

| | Process parameters | | | |
|---|---|---|---|---|
| | High temperature cycle 1 | | High temperature cycle 2 | |
| | Cycle | Cumulative | Cycle | Cumulative |
| | decription | Time, | decription | Time, |
| Process | Loading | hours | Loading | hours |
| steps | temperature | 20° C. | temperature | 20° C. |
| Freezing | Ramp to −45° C. | 2.17 | Ramp to −45° C. | 2.17 |
| including | in 130 min | | in 130 min | |
| Annealing | Hold at −45° C. | 3.17 | Hold at −48° C. | 3.17 |
| | for 60 min | | for 60 min | |
| | Ramp to −10° C. | 4.33 | Ramp to −10° C. | 4.33 |
| | in 70 min | | in 70 min | |
| | Hold at −10° C. | 7.33 | Hold at −10° C. | 7.33 |
| | for 180 min | | for 180 min | |
| | Ramp to −45° C. | 8.50 | Ramp to −45° C. | 8.50 |
| | in 70 min | | in 70 min | |
| | Hold at −45° C. | 9.50 | Hold at −45° C. | 9.50 |
| | for 60 min | | for 60 min | |
| Chill | Temperature_{condenser} | 10.50 | Temperature_{condenser} | 10.50 |
| condenser | <−60° C.; | | <−60° C.; | |
| and pull | Pressure_{chamber} = | | Pressure_{chamber} = | |
| vacuum | 50 mTorr | | 50 mTorr | |
| Primary | Ramp to −20° C. | 11.33 | Ramp to −15° C. | 11.50 |
| drying | in 50 min | | in 60 min | |
| | Hold at −20° C. | 23.33 | Hold at −15° C. | 12.75 |
| | for 720 min | | for 75 min, | |
| | | | Pch = 50 mT | |
| | — | — | Hold at −15° C. | 16.50 |
| | | | for 225 min, | |
| | | | Pch = 45 mT | |
| | — | — | Hold at −15° C. | 21.50 |
| | | | for 300 min, | |
| | | | Pch = 40 mT | |
| Secondary | Ramp to 30° C. in | 27.50 | Ramp to32° C. | 25.25 |
| | 250 min | | in 300 min | |
| drying | Hold at 30° C. for | 35.50 | Hold at 32° C. | 33.25 |
| | 480 min | | for 3600 min | |

### Example 3

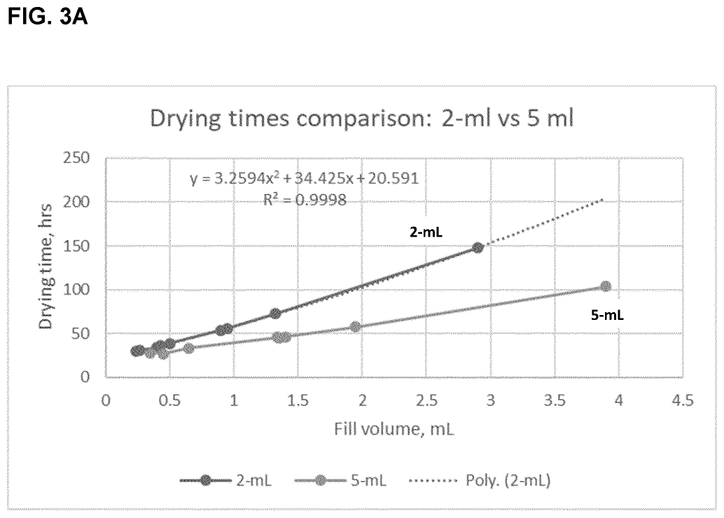

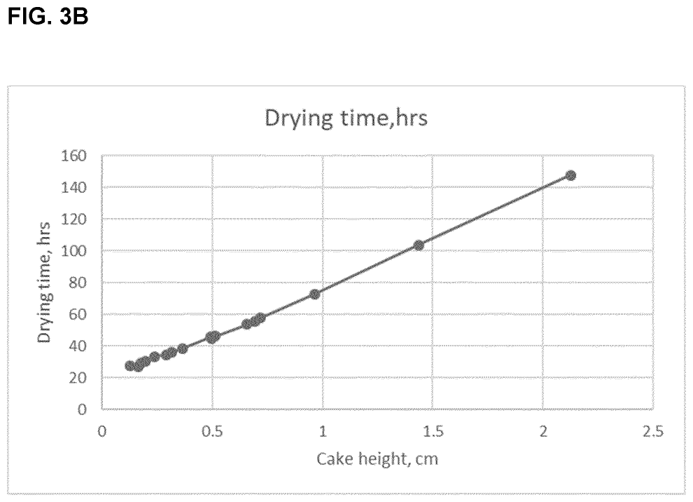

In this Example, applicant demonstrates the relationship between fill volume and drying time for different container size (e.g., 2-mL vial versus 5-mL vial) (FIG. 3A). Applicant also demonstrates the impact of cake height on drying time for any size vial (FIG. 3B). Note that the relationships shown in FIGS. 3A and 3B are exemplary and can vary depending on the glass transition temperature (Tg′) of the formulations. Tg′ of approximately −33° C. was used to establish these relationships for formulations F9-F16 shown in FIG. 3A-3B.

### Example 4: Formulations for Freeze-Dried Drug Products

Three formulations were investigated:
i) Sucrose-PBS formulation,
ii) Trehalose-Tris-NaCl formulation, and
iii) Mannitol-Sucrose-Tris-NaCl formulation.

Additional formulation screening studies showed that removal of NaCl from formulation did not result in a negative impact on the product quality attributes and also supported the development of a shorter lyophilization cycle (data not shown).

1. Formulations Screened

Sucrose and trehalose were utilized as cryo- and lyoprotectants and bulking agents for the formulation screening study. The disaccharides were utilized at a concentration of 10% w/v since it yields a nearly isotonic solution. Tris, histidine, and HEPES buffers at corresponding appropriate pH values (of 6.5 or 7.5) were included as alternatives to phosphate buffer. Approximately 10 mM of buffer is assumed to be sufficient for maintaining the pH of the candidate formulations. The impact of sodium chloride on product quality was also be investigated. The formulation compositions are summarized in Table 8.

TABLE 8
Compositions of formulations for lyophilization

| Formulation # | Formulation description |
|---|---|
| 1 | 10% w/v Sucrose + 10 mM Tris, pH 7.4 |
| 2 | 10% w/v Trehalose + 10 mM Tris, pH 7.4 |
| 3 | 5% w/v Sucrose + 5% w/v Trehalose + |
| | 10 mM Tris, pH 7.4 |
| 4 | 10% w/v Sucrose + |
| | 6 mg/mL NaCl + 10 mM Tris, pH 7.4 |
| 5 | 10% w/v Trehalose + |
| | 6 mg/mL NaCl + 10 mM Tris, pH 7.4 |
| 6 | 5% w/v Sucrose + 5% w/v Trehalose + |
| | 6 mg/mL NaCl + 10 mM Tris, pH 7.4 |
| 7 | 10% w/v Sucrose + 10 mM Histidine, pH 6.5 |
| 8 | 10% w/v Trehalose + 10 mM Histidine, pH 6.5 |
| 9 | 5% w/v Sucrose + 5% w/v Trehalose + |
| | 10 mM Histidine, pH 6.5 |
| 10 | 10% w/v Sucrose + 10 mM HEPES, pH 7.4 |
| 11 | 10% w/v Trehalose + 10 mM HEPES, pH 7.4 |
| 12 | 5% w/v Sucrose + 5% w/v Trehalose + |
| | 10 mM HEPES, pH 7.4 |
| 13 | 10% w/v Sucrose + 1.08 mg/mL Na₂HPO₄ + |
| | 0.18 mg/mL KH₂PO₄, PH 7.4 |
| 14 | 10% w/v Sucrose + 1.08 mg/mL Na₂HPO₄ + |
| | 0.18 mg/mL KH₂PO₄, PH 7.4 + |
| | 6 mg/mL NaCl + 0.15 mg/mL KCl |
| 15 | 10% w/v Sucrose + 10 mM Tris, pH 7.4 − |

2. Cycles for Lyophilization

The design of the lyophilization process is based on modeling approach and best practices in freeze-drying. Cooling and warming ramps during freezing step were performed at 0.5° C./min (achievable for all commercial freeze-dryers). The formulations were frozen to a temperature below Tg′ of formulations. An annealing temperature of −10° C. was identified to maximize Ostwald ripening during the isothermal hold (and thereby, increase the size of the ice crystals) and decrease cake resistance while keeping the product below the melting point of the formulations. The ramp rate to secondary drying was 0.2° C./min as is recommended in the literature. The inputs into the model, needed to calculate primary drying cycle parameters, are summarized in Table 9. The primary drying model is described in the art (B. Bhatnagar, S. Tchessalov, L. Lewis, and R. Johnson, Freeze-drying of biologics. Encyclopedia of Pharmaceutical Science and Technology, 4th edition, publisher Taylor & Francis, 1673-1722).

TABLE 9
Inputs into the primary drying model

| Input | | Coefficients in the equations | | | | |
|---|---|---|---|---|---|---|
| parameter | Value | for vial heat transfer (Kv) and cake resistance | | | | |
| Vial | 2- mL | Kv*10⁴, | | Resistance, Rp, Torr*hr*cm²/g | | |
| Diameterᵢₙ, | 1.4 | Cal/s/ | | | Case 1: | Case 2: |
| cm | | cm²/K | | | Calculated | Calculated |
| | | | | | for 10.3% | for 10% w/v |
| Diameterₒᵤₜ, | 1.6 | | | | w/v sucrose + | sucrose |
| cm | | | | | PBS (no | (annealing, |
| | | | | | annealing) | no NaCI). |
| Solids | 114.46 mg/ | A | 1.10499 | A | 0.566327444 | 1.146604102 |
| content | ml or 11.4% | | | | | |
| mg/ml or | (no NaCI) | | | | | |
| % | 120.46 mg | | | | | |
| | /ml or 12.0% | | | | | |
| | (with NaCI) | | | | | |
| Fill volume, | 0.5 | B | 35.24706 | B | 25.78454046 | 24.86268276 |
| mL | (corresponds to 5 | | | | | |
| | doses) | | | | | |
| Density, | 1.035 | C | 2.74684 | C | 0 | 3.685315909 |
| g/cm^{∧}3 | | | | | | |

3. Lyophilization Cycle Parameters:

The cycle parameters were calculated for:
i. two types of formulations (containing sucrose with salt (NaCl) vs. sucrose without salt), and
ii. two cycle conditions (annealing vs. no annealing).

Trehalose based formulations are assumed to be more robust to collapse when compared to sucrose and will also be co-lyophilized with more sensitive-to-collapse sucrose formulations. The critical (target temperature) used during calculations was −36° C. and −40° C. for sucrose formulations without and with sodium chloride, respectively. The cycle parameters were calculated using a model of primary drying and applying best practices described in the literature, as summarized in Table 10.

TABLE 10
Cycles designed for the formulations shown in Table 8.

| | Process parameters | | | | | | | |
|---|---|---|---|---|---|---|---|---|
| | Cycle 1 | | Cycle 2 | | Cycle 3 | | Cycle 4 | |
| | target | | target | | target | | target | |
| Process | temperature | Time, | temperature | Time, | temperature | Time, | temperature | Time, |
| steps | −40ºC | hours | −40°C | hours | −36° C. | hours | −36° C. | hours |
| Freezing | R to −45° C. in | 2.17 | R to −45° C. in | 2.17 | R to −45° C. in | 2.17 | R to −45° C. in | 2.17 |
| | 130 min | | 130 min | | 130 min | | 130 min | |
| | Hat −45° C. | 3.17 | H at −45º C. for | 3.17 | H at −45º C. for | 3.17 | Hat −45º C. for | 3.17 |
| | for 60 min | | 60 min | | 60 min | | 60 min | |
| | | | R to −10° C. in | 4.33 | | | R to −10° C. in | 4.33 |
| | | | 70 min | | | | 70 min | |
| | | | Hat −10º C. for | 7.33 | | | Hat −10º C. for | 7.33 |
| | | | 180 min | | | | 180 min | |
| | | | R to −45° C. in | 8.50 | | | R to −45° C. in | 8.50 |
| | | | 70 min | | | | 70 min | |
| | | | H at −45° C. for | 9.50 | | | H at −45º C. for | 9.50 |
| | | | 60 min | | | | 60 min | |
| Chill | Tcond < −50° C.; | 4.17 | Tcond < −50° C.; | 10.50 | Tcond < −50° C., | 4.17 | Tcond < −50° C.; | 10.50 |
| condenser | Pch = 30 mT | | Pch = 40 mT | | Pch = 40 mT | | Pch = 50 mT | |
| and pull | | | | | | | | |
| vacuum | | | | | | | | |
| Primary | R to −38º C. in | 4.40 | R to −36º C. in | 10.80 | R to −32º C. in | 4.60 | R to −29º C. in | 11.03 |
| drying | 14 min | | 18 min | | 26 min | | 32 min | |
| | Hat −38° C. | 64.40 | H at −36º C. for | 55.80 | Hat −32º C. for | 44.60 | H at −29º C. for | 41.03 |
| | for 3600 min | | 2700 min | | 2400 min | | 1800 min | |
| Secondary | R to 30° C. in | 70.07 | R to 30° C. in | 61.30 | R to 30° C. in | 49.77 | R to 30° C. in | 45.95 |
| drying | 340 min | | 330 min | | 310 min | | 295 min | |
| | H at 30º C. for | 78.07 | H at 30º C. for | 69.30 | H at 30° C. for | 57.77 | H at 30° C. for | 53.95 |
| | 480 min | | 480 min | | 480 min | | 480 min | |
| Formula- | 4-6, 14-15 | | 4-6, 14-15 | | 1-3,7-13 | | 1-3 | |
| tions to be | | | | | | | | |
| used (See | | | | | | | | |
| Table 8) | | | | | | | | |

i. R = ramp, H = hold
ii. Note that cycle parameters were calculated assuming edge effect of 1.5.

4. Stability Program

A stability study was conducted to screen the formulations as shown in Table 8.

5. Analytical Assessment of Product Quality

The analytical assays used in formulation screening are shown in Table 11.

TABLE 11
Assays for characterization of the lyophilized
drug product set forth in Example 4.
Assay

Residual moisture by Karl Fischer
DSC (high temperature)
Dynamic Light Scattering
(Z-average and PDI)
SPOS
Electron Microscopy
Encapsulation efficiency
(RiboGreen assay), %
In vitro expression (% cells positive)
In vitro expression (MFI)
Lipids content
Fragment analysis (mRNA integrity)

### Example 5: Improved Lyophilization Process

1. Cooling/freezing rate during freezing of the formulation over 0.02° C./min to 37° C./min. There was no negative impact of cooling rates on product quality.

2. Cooling/freezing rate during freeze-drying: PCS (precooled shelf) vs. 1° C./min vs. 0.5° C./min.

3. Annealing of the formulation during freezing after initial cooling to enable batch homogeneity, scale-up and tech transfer. There was no negative effect of an increased mobility during annealing on drug product quality.

4. Drying below the collapse temperature (sucrose-based formulations).

5. Drying close to the collapse temperature by changing the shelf temperature and chamber pressure (trehalose-based formulations).

6. Inclusion of combination of cryo-protectants (sucrose-trehalose, trehalose-sodium chloride) to inhibit crystallization of formulation components.

7. Inclusion of combination of components (sucrose-sorbitol) to further enhance cryo- and lyo-protection. “Cryoprotectant” refers to a component that provides stabilization during cooling/freeizng. “Lyoprotectant” refers to a component that provides stabilization during drying and in the dried state.

8. Freeze-drying of salt containing vs. salt-free formulations.

9. Freeze-drying of formulations containing low to high drug product to enable single and multiple dose vial presentations.

10. Inclusion of surfactant (e.g. polysorbates or poloxamers) to investigate the effect on colloidal stability.

11. Inclusion of a preservative (organic solvent, e.g. 2-Phenoxyethanol, m-cresol, benzyl alcohol) to enable multi-dose drug product formulation presentations.

12. Inclusion of excipients (glutathione, EDTA, DTPA, methionine, desferal) to improve stability.

13. Spray freeze-drying of drug product formulations.

14. Current fabrication vs. Alternate fabrication of drug product formulations.

### Example 6: Lyophilization Cycles for Encapsulated Pharmaceutical Substance (Commercial Cycles)

TABLE 12
Lyophilization Cycle 1 with a Target Product
Temperature of −33° C.

Process parameters

| Process | Step | Cumulative |
|---|---|---|
| steps | description | time, hrs |
| Freezing | Ramp to −45° C. | 2.17 |
| and | at 0.5° C./min | |
| annealing | Hold at −45° C. | 3.17 |
| | for 1 hour | |
| | Ramp to −10° C. | 4.33 |
| | at 0.5° C./min | |
| | Hold at −10° C. | 7.33 |
| | for 3 hours | |
| | Ramp to −45° C. | 8.50 |
| | at 0.5 C./min | |
| | Hold at −45° C. | 9.50 |
| | for 1 hour | |
| Condenser cooling | Condenser temperature | 10.50 |
| and vacuum | below −60° C., | |
| initiation | pressure 50 mT | |
| Primary | Ramp to −20° C. | 11.33 |
| drying | at 0.5 C./min, | |
| | P = 50 mT | |
| | Hold at −20° C. | 23.33 |
| | for 12 hours, | |
| | P = 50 mT | |
| Secondary | Ramp to 30° C. | 27.50 |
| drying | at 0.2 C./min, | |
| | P = 50 mT | |
| | Hold at 30° C. | 35.50 |
| | for 8 hours | |

TABLE 13
Lyophilization Cycle 2 with a Target Product
Temperature of −33° C. or higher

Process parameters

| Process | Step | Cumulative |
|---|---|---|
| steps | description | time, hrs |
| Freezing and | Ramp to −45° C. | 2.17 |
| annealing | at 0.5 C/min | |
| | Hold at −45° C. | 3.17 |
| | for 1 hour | |
| | Ramp to −10° C. | 4.33 |
| | at 0.5° C./mi | |
| | Hold at −10° C. | 7.33 |
| | for 3 hours | |
| | Ramp to −45° C. | 8.50 |
| | at 0.5° C./min | |
| | Hold at −45° C. | 9.50 |
| | for 1 hour | |
| Condenser cooling | Condenser temperature | 10.50 |
| and vacuum | below −60° C., | |
| initiation | pressure 50 mT | |
| Primary | Ramp to −15° C. | 11.50 |
| drying | at 0.5° C./min, | |
| | P = 50 mT | |
| | Hold at −15° C. | 12.50 |
| | for 1 hour, | |
| | P = 50 mT | |
| | Hold at −15° C. | 16.50 |
| | for 4 hours, | |
| | P = 45 mT | |
| | Hold at −15° C. | 21.50 |
| | for 5 hours, | |
| | P = 40 mT | |
| Secondary | Ramp to 30° C. | 25.25 |
| drying | at 0.2° C./min, | |
| | P = 50 mT | |
| | Hold at 30° C. | 33.25 |
| | for 8 hours | |

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method for lyophilizing a liquid formulation, wherein the method comprises the following steps:
a) providing a liquid formulation comprising a pharmaceutical substance and a stabilizing agent, wherein the liquid formulation comprises a specific ratio of the pharmaceutical substance to the stabilizing agent;
b) initiating a freezing step in order to obtain a frozen liquid by:
i) introducing the liquid formulation provided in step (a) into a freeze drying chamber of a freeze dryer; and
ii) cooling the liquid formulation to a freezing temperature, wherein the cooling is performed at a defined cooling rate in order to obtain a frozen liquid;
c) initiating a primary drying step of the frozen liquid obtained in step (b) in order to obtain a partly dried product by:
i) reducing the pressure in the freeze drying chamber to a pressure below the vapor pressure of ice, and
ii) increasing the shelf temperature;
d) initiating a secondary drying step of the partly dried product obtained in step (c) comprising heating the partly dried product obtained in step (c) to a drying temperature wherein the heating is performed at a defined heating rate in order to obtain a lyophilized composition comprising the pharmaceutical substance and the stabilizing agent; and
e) equilibrating the pressure in the freeze drying chamber to atmospheric pressure and removing the lyophilized composition comprising the pharmaceutical substance and the stabilizing agent obtained in step (d) from the freeze-drying chamber.

2. The method of claim 1, further comprising an annealing step after the initial freezing set forth in step (b).

3. The method of claim 1 or 2, wherein the lyophilized product comprises amorphous formulation components.

4. The method of any one of claims 1-3, wherein the lyophilized product comprises partially crystalline or amorphous formulation components.

5. The method of any of claims 1-4, wherein the shelf temperature during primary drying set forth in step (c) is from about −15° C. to about −30° C.

6. The method of claim 5, wherein the shelf temperature is −25° C.

7. The method of any of claims 1-6, wherein the chamber pressure during primary drying set forth in step (c) is from about 25 mTorr to about 100 mTorr.

8. The method of claim 7, wherein the chamber pressure is 50 mTorr.

9. The method of any of claims 1-8, wherein the shelf temperature during initial freezing set forth in step (b) is from about −30° C. to −60° C.

10. The method of any of claims 1-9, wherein the annealing temperature is from about −5° C. to about −25° C.

11. The method of claim 10, wherein the annealing temperature is −10° C.

12. A method for producing a stable liquid formulation comprising a mixture of a pharmaceutical substance and a stabilizing agent, wherein the method comprises mixing the pharmaceutical substance and the stabilizing agent in a specific ratio, so as to thereby produce the stable formulation.

13. The method of claim 12, wherein the stable formulation further comprises at least one encapsulating agent.

14. The method of any one of claims 12-13, wherein the stable formulation further comprises a buffer.

15. The method of any one of claims 12-14, wherein the stable formulation further comprises a salt.

16. The method of any one of claims 12-15, wherein the stable formulation further comprises a surfactant, a preservative, any other excipient, or a combination thereof.

17. The method of claim 16, wherein the surfactant, preservative, excipient or combination thereof is selected from sterile water for injection (sWFI), bacteriostatic water for injection (BWFI), saline, dextrose solution, polysorbates, poloxamers, Triton, divalent cations, Ringer's lactate, amino acids, sugars, polyols, polymers or cyclodextrins.

18. The method of any one of claims 12-17, wherein the pharmaceutical substance is selected from the group consisting of a protein, a peptide, a polysaccharide, a small molecule, a natural product, a nucleic acid, an immunogen, a vaccine, a polymer, a chemical compound, and a combination thereof.

19. The method of claim 18, wherein the pharmaceutical substance is a nucleic acid.

20. The method of claim 19, wherein the nucleic acid is selected from the group consisting of DNA, RNA, RNA/DNA hybrids, and aptamers.

21. The method of claim 20, wherein the RNA is mRNA.

22. The method of any one of claims 12-21, wherein the pharmaceutical substance concentration is less than about 0.05 mg/ml.

23. The method of any one of claims 12-21, wherein the pharmaceutical substance concentration is at least about 0.05 mg/ml.

24. The method of any one of claims 12-21, wherein the pharmaceutical substance concentration is at least about 0.5 mg/ml.

25. The method of any one of claims 12-21, wherein the pharmaceutical substance concentration is at least about 1 mg/ml.

26. The method of any one of claims 12-21, wherein the pharmaceutical substance concentration is at least about 10 mg/ml.

27. The method of any one of claims 12-21, wherein the pharmaceutical substance concentration is at least about 50 mg/ml.

28. The method of any one of claims 12-21, wherein the pharmaceutical substance concentration is from about 0.05 mg/ml to about 0.5 mg/ml.

29. The method of any one of claims 12-28, wherein the stabilizing agent is selected from the group consisting of sucrose, mannose, sorbitol, raffinose, trehalose, mannitol, inositol, sodium chloride, arginine, lactose, hydroxyethyl starch, dextran, polyvinylpyrolidone, glycine, and a combination thereof.

30. The method of claim 29, wherein the stabilizing agent is sucrose.

31. The method of any one of claims 12-30, wherein the stabilizing agent concentration is from about 100 mg/mL to about 200 mg/mL.

32. The method of any one of claims 12-31, wherein the pharmaceutical substance is mRNA and the stabilizing agent is sucrose.

33. The method of any one of claims 12-32, wherein the ratio of the mass amount of the stabilizing agent and the pharmaceutical substance is no greater than 5000.

34. The method of any one of claims 12-32, wherein the ratio of the mass amount of the stabilizing agent and the pharmaceutical substance is no greater than 2000.

35. The method of any one of claims 12-32, wherein the ratio of the mass amount of the stabilizing agent and the pharmaceutical substance is no greater than 1000.

36. The method of any one of claims 12-32, wherein the ratio of the mass amount of the stabilizing agent and the pharmaceutical substance is no greater than 500.

37. The method of any one of claims 12-32, wherein the ratio of the mass amount of the stabilizing agent and the pharmaceutical substance is no greater than 100.

38. The method of any one of claims 12-32, wherein the ratio of the mass amount of the stabilizing agent and the pharmaceutical substance is no greater than 50.

39. The method of any one of claims 12-32, wherein the ratio of the mass amount of the stabilizing agent and the pharmaceutical substance is no greater than 10.

40. The method of any one of claims 12-32, wherein the ratio of the mass amount of the stabilizing agent and the pharmaceutical substance is no greater than 1.

41. The method of any one of claims 12-32, wherein the ratio of the mass amount of the stabilizing agent and the pharmaceutical substance is no greater than 0.5.

42. The method of any one of claims 12-32, wherein the ratio of the mass amount of the stabilizing agent and the pharmaceutical substance is no greater than 0.1.

43. The method of any one of claims 12-32, wherein the stabilizing agent and pharmaceutical substance comprise a mass ratio of about 200-2000 of the stabilizing agent: 1 of the pharmaceutical substance.

44. The method of any one of claims 13-43, wherein the encapsulating agent is selected from the group consisting of a lipid, a lipid nanoparticle (LNP), lipoplexes, polymeric particles, polyplexes, and monolithic delivery systems, and a combination thereof.

45. The method of claim 44, wherein the encapsulating agent is a lipid nanoparticle (LNP).

46. The method of any one of claims 12-45, wherein the stable formulation is stored at ambient temperature or subambient temperature for a defined period.

47. A method for preparing a stable formulation according to the method of claim 1 or 12, wherein the formulation is stable as a frozen matrix, a refrigerated liquid, or a refrigerated lyophilized product.

48. The method of claim 47, wherein the formulation is above the glass transition temperature of the frozen matrix (Tg′).

49. The method of claim 48, wherein the formulation remains amorphous during isothermal hold or storage above Tg′.

50. The method of claim 47, wherein the storage temperature is ambient temperature or below ambient temperature

51. A method of improving the stability of the lyophilized composition of any of claims 1-11, and 47-50 by adding glutathione, EDTA, methionine, desferal and any antioxidants or metal scavengers to the liquid formulation prior to lyophilization or during reconstitution of the lyophilized composition in a form suitable for injection.

52. The method of claims 1-11, and 47-51, wherein the formulation further comprises at least one encapsulating agent.

53. The method of any one of claims 1-11, and 47-52, wherein the formulation further comprises a buffer.

54. The method of any one of claims 1-11, and 47-53, wherein the formulation further comprises a salt.

55. The method of any one of claims 1-11, and 47-54, wherein the formulation further comprises a surfactant, a preservative, any other excipient, or a combination thereof.

56. The method of claim 55, wherein the surfactant, preservative, excipient or combination thereof is selected from sterile water for injection (sWFI), bacteriostatic water for injection (BWFI), saline, dextrose solution, polysorbates, poloxamers, Triton, divalent cations, Ringer's lactate, amino acids, sugars, polyols, polymers or cyclodextrins.

57. The method of any one of claims 1-11, and 47-56, wherein the pharmaceutical substance is selected from the group consisting of a protein, a peptide, a polysaccharide, a small molecule, a natural product, a nucleic acid, an immunogen, a vaccine, a polymer, a chemical compound, and a combination thereof.

58. The method of claim 57, wherein the pharmaceutical substance is a nucleic acid.

59. The method of claim 58, wherein the nucleic acid is selected from the group consisting of DNA, RNA, RNA/DNA hybrids, and aptamers.

60. The method of claim 59, wherein the RNA is mRNA.

61. The method of any one of claims 1-11, and 47-60, wherein the pharmaceutical substance concentration is less than about 0.05 mg/ml.

62. The method of any one of claims 1-11, and 47-60, wherein the pharmaceutical substance concentration is at least about 0.05 mg/ml.

63. The method of any one of claims 1-11, and 47-60, wherein the pharmaceutical substance concentration is at least about 0.5 mg/ml.

64. The method of any one of claims 1-11, and 47-60, wherein the pharmaceutical substance concentration is at least about 1 mg/ml.

65. The method of any one of claims 1-11, and 47-60, wherein the pharmaceutical substance concentration is at least about 10 mg/ml.

66. The method of any one of claims 1-11, and 47-60, wherein the pharmaceutical substance concentration is at least about 50 mg/ml.

67. The method of any one of claims 1-11, and 47-60, wherein the pharmaceutical substance concentration is from about 0.05 mg/ml to about 0.5 mg/ml.

68. The method of any one of claims 1-11, and 47-67, wherein the stabilizing agent is selected from the group consisting of sucrose, mannose, sorbitol, raffinose, trehalose, mannitol, inositol, sodium chloride, arginine, lactose, hydroxyethyl starch, dextran, polyvinylpyrolidone, glycine, and a combination thereof.

69. The method of claim 68, wherein the stabilizing agent is sucrose.

70. The method of any one of claims 1-11, and 47-69, wherein the stabilizing agent concentration is from about 100 mg/mL to about 200 mg/mL.

71. The method of any one of claims 1-11, and 47-70, wherein the pharmaceutical substance is mRNA and the stabilizing agent is sucrose.

72. The method of any one of claims 1-11, and 47-71, wherein the ratio of the mass amount of the stabilizing agent and the pharmaceutical substance is no greater than 5000.

73. The method of any one of claims 1-11, and 47-71, wherein the ratio of the mass amount of the stabilizing agent and the pharmaceutical substance is no greater than 2000.

74. The method of any one of claims 1-11, and 47-71, wherein the ratio of the mass amount of the stabilizing agent and the pharmaceutical substance is no greater than 1000.

75. The method of any one of claims 1-11, and 47-71, wherein the ratio of the mass amount of the stabilizing agent and the pharmaceutical substance is no greater than 500.

76. The method of any one of claims 1-11, and 47-71, wherein the ratio of the mass amount of the stabilizing agent and the pharmaceutical substance is no greater than 100.

77. The method of any one of claims 1-11, and 47-71, wherein the ratio of the mass amount of the stabilizing agent and the pharmaceutical substance is no greater than 50.

78. The method of any one of claims 1-11, and 47-71, wherein the ratio of the mass amount of the stabilizing agent and the pharmaceutical substance is no greater than 10.

79. The method of any one of claims 1-11, and 47-71, wherein the ratio of the mass amount of the stabilizing agent and the pharmaceutical substance is no greater than 1.

80. The method of any one of claims 1-11, and 47-71, wherein the ratio of the mass amount of the stabilizing agent and the pharmaceutical substance is no greater than 0.5.

81. The method of any one of claims 1-11, and 47-71, wherein the ratio of the mass amount of the stabilizing agent and the pharmaceutical substance is no greater than 0.1.

82. The method of any one of claims 1-11, and 47-71, wherein the stabilizing agent and pharmaceutical substance comprise a mass ratio of about 200-2000 of the stabilizing agent: 1 of the pharmaceutical substance.

83. The method of any one of claims 52-82, wherein the encapsulating agent is selected from the group consisting of a lipid, a lipid nanoparticle (LNP), lipoplexes, polymeric particles, polyplexes, and monolithic delivery systems, and a combination thereof.

84. The method of claim 83, wherein the encapsulating agent is a lipid nanoparticle (LNP).

85. The method of any one of claims 1-11, and 47-84, wherein the stable formulation is stored at ambient temperature or subambient temperature for a defined period.

86. A method for preparing a stable formulation comprising the steps of:
(a) lyophilizing a mixture of a pharmaceutical substance and a stabilizing amount of a stabilizing agent which prevents or reduces chemical or physical instability of the pharmaceutical substance upon lyophilization and subsequent storage as set forth in any one of claims 1-11, and 47-85, and
(b) reconstituting the lyophilized mixture of step (a) in a diluent such that the reconstituted formulation is stable.

87. The method of claim 86, wherein the diluent is selected from sterile water for injection (sWFI), saline, dextrose solution, polysorbates, poloxamers, Triton, divalent cations, Ringer's lactate, amino acids, sugars, polyols, polymers or cyclodextrins, pH buffered diluents, or preservative containing diluents such as bacteriostatic water for injection (BWFI), 2-phenoxyethanol, m-cresol, or phenol.

88. A method of storing a pharmaceutical substance comprising the steps of:
(a) producing a stable formulation according to the method of any one of claims 12-46 or a lyophilized composition of any of claims 1-11, and 47-85; and
(b) storing the stable formulation or lyophilized composition for a defined period.

89. The method of claim 54, wherein the period is longer than 3 months.

90. The method of claim 54, wherein the period is longer than 8 months.

91. The method of claim 54, wherein the period is longer than 12 months.

92. The method of claim 54, wherein the period is longer than 18 months.

93. The method of claim 54, wherein the period is longer than 24 months.

94. A stable formulation produced using the method of any one of claims 12-46.

95. A lyophilized composition produced using the method of any one of claims 1-11, and 47-85.

96. The lyophilized composition of claim 95, wherein the lyophilized composition has a cake height of up to 3 cm.
